SEQUENCE LISTING (63 sequences)

SEQ ID NO: 1 - RNA, 204 nt, synthetic construct
  source 1..204: mol_type = other RNA
  misc_feature 3..4: note = RNA
catttgcgtgggttcagacagcccccgggggacgccactcggttccggccagaatcgggcctcaagaacttagacgcgcgcataccagccctaacggaccaccaggtgttccagcccgcttgggtaccgtgcgcagcggcggccggggtgtgtcaagcgacagagggtcggccgagcaggcgcagccccggcgcaggttacgcc

SEQ ID NO: 2 - RNA, 204 nt, synthetic construct
  source 1..204: mol_type = other RNA
aacagagcacattaccagaagctattttgtgcgttagtttgctttaacggtaccttttaattaaacatacaagcaatttagtatatattctataatattaacacaatatagcatactacgtaattttaagctacaaaattgttcagttggtgtacaagtggtcatcgcgccatacgacatattcgacatatcttactcatcgcc

SEQ ID NO: 3 - RNA, 204 nt, synthetic construct
  source 1..204: mol_type = other RNA
ttcccgggacccggtcgtgggttcgagtcacctaaaggagcaaaagcgtgttattgtagtttcgaggcacggaactcaataccattacggtgaaccccacgaaccttcgacgtttgggcatatatattctcacgcagaaagtcaggataggacttgcgggtatacagtgccagccgcttcgctctactttaactaacgatcgcc

SEQ ID NO: 4 - RNA, 204 nt, synthetic construct
  source 1..204: mol_type = other RNA
catttgcgtgggttcagacagcccccgggggacgccactcggttccggccagaatcgggcctcaagaacttagacgcgcgcataccagccctaacggaccaccaggtgttccagcccgcttgggtaccgtgcgcagcggcggccggggtgtgtcaagcgacagagggtcggccgagcaggcgcagccccggcgcaggttacttt

SEQ ID NO: 5 - RNA, 19 nt, synthetic construct
  source 1..19: mol_type = other RNA
ttgggccgttaaggctcaa

SEQ ID NO: 6 - RNA, 25 nt, synthetic construct
  source 1..25: mol_type = other RNA
cggcggggccgttaaggctccgccg

SEQ ID NO: 7 - RNA, 45 nt, synthetic construct
  source 1..45: mol_type = other RNA
gtccctttcgcggcggggccgttaaggctccgccgcgaaagggac

SEQ ID NO: 8 - RNA, 20 nt, synthetic construct
  source 1..20: mol_type = other RNA
gatccgggttctcccggatc

SEQ ID NO: 9 - RNA, 29 nt, synthetic construct
  source 1..29: mol_type = other RNA
cgcggcggggccgttaaggctccgccgcg

SEQ ID NO: 10 - RNA, 22 nt, synthetic construct
  source 1..22: mol_type = other RNA
gggctgcgggcctccgcagccc

SEQ ID NO: 11 - RNA, 29 nt, synthetic construct
  source 1..29: mol_type = other RNA
gggccgtggtggagcttccaccatggccc

SEQ ID NO: 12 - RNA, 40 nt, synthetic construct
  source 1..40: mol_type = other RNA
ggggcgcgtggtggcggctgcagccgccaccacgcgcccc

SEQ ID NO: 13 - RNA, 40 nt, synthetic construct
  source 1..40: mol_type = other RNA
  misc_feature 3: note = RNA
  misc_feature 9: note = RNA
  misc_feature 15: note = RNA
  misc_feature 11: note = RNA
  misc_feature 20: note = RNA
  misc_structure 24: note = RNA
  misc_feature 30: note = RNA
  misc_feature 36: note = RNA
  misc_feature 34: note = RNA
cgtccagatctacatagagtacgtacgcgtagatctgacg

SEQ ID NO: 14 - RNA, 16 nt, synthetic construct
  source 1..16: mol_type = other RNA
  misc_feature 4: note = RNA
cgctgcgacggcagcg

SEQ ID NO: 15 - RNA, 10 nt, synthetic construct
  source 1..10: mol_type = other RNA
  misc_feature 1: note = RNA
tgcgacggca

SEQ ID NO: 16 - RNA, 22 nt, synthetic construct
  source 1..22: mol_type = other RNA
  misc_feature 5: note = RNA
cggctccgcgacggcggagccg

SEQ ID NO: 17 - RNA, 22 nt, synthetic construct
  source 1..22: mol_type = other RNA
  misc_feature 2: note = RNA
  misc_feature 5: note = RNA
  misc_feature 17: note = RNA
ctgctacgcgacggcgtagcag

SEQ ID NO: 18 - RNA, 21 nt, synthetic construct
  source 1..21: mol_type = other RNA
  misc_feature 4..6: note = RNA
  misc_feature 10: note = RNA
  misc_feature 12..13: note = RNA
  misc_feature 19..20: note = RNA
caatttccatcttggaaattg

SEQ ID NO: 19 - RNA, 21 nt, synthetic construct
  source 1..21: mol_type = other RNA
  misc_feature 4..5: note = RNA
  misc_feature 7: note = RNA
  misc_feature 10: note = RNA
  misc_feature 12..13: note = RNA
  misc_feature 16: note = RNA
  misc_feature 19..20: note = RNA
caattatcatcttggtaattg

SEQ ID NO: 20 - RNA, 21 nt, synthetic construct
  source 1..21: mol_type = other RNA
  misc_feature 4..6: note = RNA
  misc_feature 10: note = RNA
  misc_feature 12..13: note = RNA
  misc_feature 19..20: note = RNA
caatttccatcttggcaattg

SEQ ID NO: 21 - RNA, 21 nt, synthetic construct
  source 1..21: mol_type = other RNA
  misc_feature 4..5: note = RNA
  misc_structure 10: note = RNA
  misc_feature 12..13: note = RNA
  misc_feature 19..20: note = RNA
caattcccatcttgggaattg

SEQ ID NO: 22 - RNA, 20 nt, synthetic construct
  source 1..20: mol_type = other RNA
  misc_feature 4..5: note = RNA
  misc_feature 9: note = RNA
  misc_feature 11: note = RNA
  misc_feature 13..15: note = RNA
  misc_feature 18..19: note = RNA
gaattaaatctgtttaattc

SEQ ID NO: 23 - RNA, 21 nt, synthetic construct
  source 1..21: mol_type = other RNA
  misc_feature 4..5: note = RNA
  misc_feature 10: note = RNA
  misc_feature 12..13: note = RNA
  misc_feature 19..20: note = RNA
gaattaccatcttgggacttc

SEQ ID NO: 24 - RNA, 28 nt, synthetic construct
  source 1..28: mol_type = other RNA
  misc_feature 5: note = RNA
  misc_feature 15: note = RNA
  misc_feature 25: note = RNA
cggatccggccggatccggccggatccg

SEQ ID NO: 25 - RNA, 28 nt, synthetic construct
  source 1..28: mol_type = other RNA
cccaacctgccggatgcggcaggttggg

SEQ ID NO: 26 - RNA, 28 nt, synthetic construct
  source 1..28: mol_type = other RNA
  misc_feature 15: note = RNA
  misc_feature 25: note = RNA
cggacccggccggatacggccgggtccg

SEQ ID NO: 27 - RNA, 40 nt, synthetic construct
  source 1..40: mol_type = other RNA
  misc_feature 9: note = RNA
  misc_feature 11: note = RNA
  misc_feature 19: note = RNA
cgggcgcgtggtggcggctgcagccgccaccacgcgcccg

SEQ ID NO: 28 - RNA, 21 nt, synthetic construct
  source 1..21: mol_type = other RNA
  misc_feature 7: note = RNA
  misc_feature 9..10: note = RNA
  misc_feature 12: note = RNA
  misc_feature 15..16: note = RNA
  misc_feature 18..19: note = RNA
gaacagtgttctcattgttca

SEQ ID NO: 29 - RNA, 21 nt, synthetic construct
  source 1..21: mol_type = other RNA
  misc_feature 3: note = RNA
  misc_feature 9..10: note = RNA
  misc_feature 12: note = RNA
  misc_feature 19: note = RNA
gatccgggttctcccggatca

SEQ ID NO: 30 - RNA, 21 nt, synthetic construct
  source 1..21: mol_type = other RNA
  misc_feature 9..10: note = RNA
  misc_feature 12: note = RNA
gcgccgggttctcccggcgca

SEQ ID NO: 31 - RNA, 30 nt, synthetic construct
  source 1..30: mol_type = other RNA
  misc_feature 3: note = RNA
  misc_feature 13..14: note = RNA
  misc_feature 16: note = RNA
  misc_feature 27: note = RNA
gatcgcgccgggttctcccggcgcgatcaa

SEQ ID NO: 32 - DNA, 204 bp, synthetic construct
  source 1..204: mol_type = other DNA
aacagagcacattaccagaagctattttgtgcgttagtttgctttaacggtaccttttaattaaacatacaagcaatttagtatatattctataatattaacacaatatagcatactacgtaattttaagctacaaaattgttcagttggtgtacaagtggtcatcgcgccatacgacatattcgacatatcttactcatcacc

SEQ ID NO: 33 - DNA, 204 bp, synthetic construct
  source 1..204: mol_type = other DNA
catttgcgtgggttcagacagcccccgggggacgccactcggttccggccagaatcgggcctcaagaacttagacgcgcgcataccagccctaacggaccaccaggtgttccagcccgcttgggtaccgtgcgcagcggcggccggggtgtgtcaagcgacagagggtcggccgagcaggcgcagccccggcgcaggttacacc

SEQ ID NO: 34 - DNA, 204 bp, synthetic construct
  source 1..204: mol_type = other DNA
catttgcgtgggttcagacagcccccgggggacgccactcggttccggccagaatcgggcctcaagaacttagacgcgcgcataccagccctaacggaccaccaggtgttccagcccgcgccatggccgtgcggaacagtgttctcattgttcaaagcgacagagggtcggccgagcaggcgcagccccggcgcaggttacacc

SEQ ID NO: 35 - DNA, 204 bp, synthetic construct
  source 1..204: mol_type = other DNA
catttgcgtgggttcagacagcccccgggggacgccactcggttccggccagaatcgggcctcaagaacttagacgcgcgcataccagccctaacggaccaccaggtgttccagccgccatgggtaccgtgcggaacagtgttctcattgttcaaagcgacagagggtcggccgagcaggcgcagccccggcgcaggttacacc

SEQ ID NO: 36 - DNA, 204 bp, synthetic construct
  source 1..204: mol_type = other DNA
catttgcgtgggttcagacagcccccgggggacgccactcggttccggccagaatcgggcctcaagaacttagacgcgcgcataccagccctaacggaccaccaggtgttccagccatggtgggtaccgtgcggaacagtgttctcattgttcaaagcgacagagggtcggccgagcaggcgcagccccggcgcaggttacacc

SEQ ID NO: 37 - DNA, 204 bp, synthetic construct
  source 1..204: mol_type = other DNA
catttgcgtgggttcagacagcccccgggggacgccactcggttccggccagaatcgggcctcaagaacttagacgcgcgcataccagccctaacggaccaccaggtgttgccatgggcttgggtaccgtgcggaacagtgttctcattgttcaaagcgacagagggtcggccgagcaggcgcagccccggcgcaggttacacc

SEQ ID NO: 38 - DNA, 204 bp, synthetic construct
  source 1..204: mol_type = other DNA
catttgcgtgggttcagacagcccccgggggacgccactcggttccggccagaatcgggcctcaagaacttagacgcgcgcataccagccctaacggaccaccaggtgccatggcccgcttgggtaccgtgcggaacagtgttctcattgttcaaagcgacagagggtcggccgagcaggcgcagccccggcgcaggttacacc

SEQ ID NO: 39 - DNA, 204 bp, synthetic construct
  source 1..204: mol_type = other DNA
catttgcgtgggttcagacagcccccgggggacgccactcggttccggccagaatcgggcctcaagaacttagacgcgcgcataccagccctaacggaccagccatggttccagcccgcttgggtaccgtgcggaacagtgttctcattgttcaaagcgacagagggtcggccgagcaggcgcagccccggcgcaggttacacc

SEQ ID NO: 40 - DNA, 204 bp, synthetic construct
  source 1..204: mol_type = other DNA
catttgcgtgggttcagacagcccccgggggacgccactcggttccggccagaatcgggcctcaagaacttagacgcgcgcataccagccctaacgccatggcaggtgttccagcccgcttgggtaccgtgcggaacagtgttctcattgttcaaagcgacagagggtcggccgagcaggcgcagccccggcgcaggttacacc

SEQ ID NO: 41 - DNA, 204 bp, synthetic construct
  source 1..204: mol_type = other DNA
catttgcgtgggttcagacagcccccgggggacgccactcggttccggccagaatcgggcctcaagaacttagacgcgcgcataccagcgccatgggaccaccaggtgttccagcccgcttgggtaccgtgcggaacagtgttctcattgttcaaagcgacagagggtcggccgagcaggcgcagccccggcgcaggttacacc

SEQ ID NO: 42 - DNA, 204 bp, synthetic construct
  source 1..204: mol_type = other DNA
catttgcgtgggttcagacagcccccgggggacgccactcggttccggccagaatcgggcctcaagaacttagacgcgcgcataccagccctaacggaccaccaggtgttccagcccgcttgggtgccatggggatccgggttctcccggatcaaagcgacagagggtcggccgagcaggcgcagccccggcgcaggttacacc

SEQ ID NO: 43 - DNA, 204 bp, synthetic construct
  source 1..204: mol_type = other DNA
catttgcgtgggttcagacagcccccgggggacgccactcggttccggccagaatcgggcctcaagaacttagacgcgcgcataccagccctaacggaccaccaggtgttccagcccgcgccatggccgtgcggatccgggttctcccggatcaaagcgacagagggtcggccgagcaggcgcagccccggcgcaggttacacc

SEQ ID NO: 44 - DNA, 249 bp, synthetic construct
  source 1..249: mol_type = other DNA
ttcccgggacccggtcgtgggttcgagtcacctaaaggagcaaaagcgtgttattgtagtttcgaggcacggaactcaataccattacggtgaaccccacgaaccttcgacgtttgggcatatatattctcacgcagaaagtcaggataggacttgcgggtatacagtgccagcgtcctttcgcggcggggccgttaaggctccgccgcgaaaggatgccgcttcgctctactttaactaacgatcgcc

SEQ ID NO: 45 - DNA, 296 bp, synthetic construct
  source 1..296: mol_type = other DNA
taatacgactcactatagggttcccgggacccggtcgtgggttcgagtcacctaaaggagcaaaagcgtgttattgtagtttcgaggcacggaactcaataccattacggtgaaccccacgaaccttcgacgtttgggcatatatattctcacgcagaaagtcaggataggacttgcgggtatacagtgccagcgtcctttcgcggcggggccgttaaggctccgccgcgaaaggatgccgcttcgctctactttaactaacgatcgccatggattacaaggatgacgatgacaag

SEQ ID NO: 46 - DNA, 249 bp, synthetic construct
  source 1..249: mol_type = other DNA
ttcccgggacccggtcgtgggttcgagtcacctaaaggagcaaaagcgtgttattgtagtttcgaggcacggaactcaataccattacggtgaaccccacgaaccttcgacgtttgggcatatatattctcacgcagaaagtcaggataggacttgcgggtatacagtgccagcgtcctttcgcggcggggccgttaaggctccgccgcgaaaggatgccgcttcgctagactttaactaacgatcgcc

SEQ ID NO: 47 - DNA, 296 bp, synthetic construct
  source 1..296: mol_type = other DNA
taatacgactcactatagggttcccgggacccggtcgtgggttcgagtcacctaaaggagcaaaagcgtgttattgtagtttcgaggcacggaactcaataccattacggtgaaccccacgaaccttcgacgtttgggcatatatattctcacgcagaaagtcaggataggacttgcgggtatacagtgccagcgtcctttcgcggcggggccgttaaggctccgccgcgaaaggatgccgcttcgctagactttaactaacgatcgccatggattacaaggatgacgatgacaag

SEQ ID NO: 48 - DNA, 249 bp, synthetic construct
  source 1..249: mol_type = other DNA
ttcccgggacccggtcgtgggttcgagtcacctaaaggagcaaaagcgtgttattgtagtttcgaggcacggaactcaataccattacggtgaaccccacgaaccttcgacgtttgggcatatatattctcacgcagaaagtcaggataggacttgcgggtatacagtgccagcgtctgtacttcgtgtggggagaggccctacgtgaagtgtagatgccgcttcgctagactttaactaacgatcgcc

SEQ ID NO: 49 - DNA, 296 bp, synthetic construct
  source 1..296: mol_type = other DNA
taatacgactcactatagggttcccgggacccggtcgtgggttcgagtcacctaaaggagcaaaagcgtgttattgtagtttcgaggcacggaactcaataccattacggtgaaccccacgaaccttcgacgtttgggcatatatattctcacgcagaaagtcaggataggacttgcgggtatacagtgccagcgtctgtacttcgtgtggggagaggccctacgtgaagtgtagatgccgcttcgctagactttaactaacgatcgccatggattacaaggatgacgatgacaag

SEQ ID NO: 50 - DNA, 249 bp, synthetic construct
  source 1..249: mol_type = other DNA
ttcccgggacccggtcgtgggttcgagtcacctaaaggagcaaaagcgtgttattgtagtttcgaggcacggaactcaataccattacggtgaaccccacgaaccttcgacgtttgggcatatatattctcacgcagaaagtcaggataggacttgcgggtatacagtgccagcgtctgtacttcgtgtggggagaggccctacgtgaagtgtagatgccgcttcgctctactttaactaacgatcgcc

SEQ ID NO: 51 - DNA, 296 bp, synthetic construct
  source 1..296: mol_type = other DNA
taatacgactcactatagggttcccgggacccggtcgtgggttcgagtcacctaaaggagcaaaagcgtgttattgtagtttcgaggcacggaactcaataccattacggtgaaccccacgaaccttcgacgtttgggcatatatattctcacgcagaaagtcaggataggacttgcgggtatacagtgccagcgtctgtacttcgtgtggggagaggccctacgtgaagtgtagatgccgcttcgctctactttaactaacgatcgccatggattacaaggatgacgatgacaag

SEQ ID NO: 52 - DNA, 249 bp, synthetic construct
  source 1..249: mol_type = other DNA
ttcccgggacccggtcgtgggttcgagtcacctaaaggagcaaaagcgtgttattgtagtttcgaggcacggaactcaataccattacggtgaaccccacggtcatgttcgcggcggggccgttaaggctccgccgcgaacgtgacaaccttcgacgtttgggcatatatattctcacgcagaaagtcaggataggacttgcgggtatacagtgccagccgcttcgctctactttaactaacgatcgcc

SEQ ID NO: 53 - DNA, 296 bp, synthetic construct
  source 1..296: mol_type = other DNA
taatacgactcactatagggttcccgggacccggtcgtgggttcgagtcacctaaaggagcaaaagcgtgttattgtagtttcgaggcacggaactcaataccattacggtgaaccccacggtcatgttcgcggcggggccgttaaggctccgccgcgaacgtgacaaccttcgacgtttgggcatatatattctcacgcagaaagtcaggataggacttgcgggtatacagtgccagccgcttcgctctactttaactaacgatcgccatggcggccgctgattacaaggatgac

SEQ ID NO: 54 - DNA, 249 bp, synthetic construct
  source 1..249: mol_type = other DNA
catttgcgtgggttcagacagcccccgggggacgccactcggttccggccagaatcgggcctcaagaacttagacgcgcgcataccagccctaacggaccacccgcccccctcttgttaacttatggtggggagatagcctaccgtacaggtgttccagcccgcttgggtaccgtgcgcagcggcggccggggtgtgtcaagcgacagagggtcggccgagcaggcgcagccccggcgcaggttacgcc

SEQ ID NO: 55 - DNA, 296 bp, synthetic construct
  source 1..296: mol_type = other DNA
taatacgactcactatagggcatttgcgtgggttcagacagcccccgggggacgccactcggttccggccagaatcgggcctcaagaacttagacgcgcgcataccagccctaacggaccacccgcccccctcttgttaacttatggtggggagatagcctaccgtacaggtgttccagcccgcttgggtaccgtgcgcagcggcggccggggtgtgtcaagcgacagagggtcggccgagcaggcgcagccccggcgcaggttacgccatggcggccgctgattacaaggatgac

SEQ ID NO: 56 - DNA, 244 bp, synthetic construct
  source 1..244: mol_type = other DNA
acagccgtcctttcgcggcggggccgttaaggctccgccgcgaaaggatggcgcggggctagccagcgagagggcgcgagcggcggcgctgcctgcagcctgcagcctgcagcctccggccggccggcgagccagtgcgcgtgcgcggcggcggcctccgcagcgaccggggagcggactgaccggcgggagggctagcgagccagcggtgtgaggcgcgaggcgaggccgagccgcgagcgac

SEQ ID NO: 57 - DNA, 291 bp, synthetic construct
  source 1..291: mol_type = other DNA
taatacgactcactatagggacagccgtcctttcgcggcggggccgttaaggctccgccgcgaaaggatggcgcggggctagccagcgagagggcgcgagcggcggcgctgcctgcagcctgcagcctgcagcctccggccggccggcgagccagtgcgcgtgcgcggcggcggcctccgcagcgaccggggagcggactgaccggcgggagggctagcgagccagcggtgtgaggcgcgaggcgaggccgagccgcgagcgacatggcggccgctgattacaaggatgac

SEQ ID NO: 58 - DNA, 244 bp, synthetic construct
  source 1..244: mol_type = other DNA
acaggcaatgtacttcgtgtagggagaggccctacgtgaagtgcgttgccgcgcggggcgagccagcgagagggcgcgagcggcggcgctgcctgcagcctgcagcctgcagcctccggccggccggcgagccagtgcgcgtgcgcggcggcggcctccgcagcgaccggggagcggactgaccggcgggagggctagcgagccagcggtgtgaggcgcgaggcgaggccgagccgcgagcgac

SEQ ID NO: 59 - DNA, 291 bp, synthetic construct
  source 1..291: mol_type = other DNA
taatacgactcactatagggacaggcaatgtacttcgtgtagggagaggccctacgtgaagtgcgttgccgcgcggggcgagccagcgagagggcgcgagcggcggcgctgcctgcagcctgcagcctgcagcctccggccggccggcgagccagtgcgcgtgcgcggcggcggcctccgcagcgaccggggagcggactgaccggcgggagggctagcgagccagcggtgtgaggcgcgaggcgaggccgagccgcgagcgacatggcggccgctgattacaaggatgac

SEQ ID NO: 60 - DNA, 244 bp, synthetic construct
  source 1..244: mol_type = other DNA
acaggtcatgttcgcggcgtagccgttaaggctccgccgcgaacgtgaccgcgcggggcgagccagcgagagggcgcgagcggcggcgctgcctgcagcctgcagcctgcagcctccggccggccggcgagccagtgcgcgtgcgcggcggcggcctccgcagcgaccggggagcggactgaccggcgggagggctagcgagccagcggtgtgaggcgcgaggcgaggccgagccgcgagcgac

SEQ ID NO: 61 - DNA, 291 bp, synthetic construct
  source 1..291: mol_type = other DNA
taatacgactcactatagggacaggtcatgttcgcggcgtagccgttaaggctccgccgcgaacgtgaccgcgcggggcgagccagcgagagggcgcgagcggcggcgctgcctgcagcctgcagcctgcagcctccggccggccggcgagccagtgcgcgtgcgcggcggcggcctccgcagcgaccggggagcggactgaccggcgggagggctagcgagccagcggtgtgaggcgcgaggcgaggccgagccgcgagcgacatggcggccgctgattacaaggatgac

SEQ ID NO: 62 - DNA, 249 bp, synthetic construct
  source 1..249: mol_type = other DNA
cattgtccctttcgcggcggggccgttaaggctccgccgcgaaagggactgcgtgggttcagacagcccccgggggacgccactcggttccggccagaatcgggccgtcttacaacttaccatttatgctcccacaatagatccgctccgctcatccagcccgcttgggtaccgtgcgcagcggcggccggggtgtgtcaagcgacagagggtcggccgagcaggcgcagccccggcgcaggttacgcc

SEQ ID NO: 63 - DNA, 296 bp, synthetic construct
  source 1..296: mol_type = other DNA
taatacgactcactatagggcattgtccctttcgcggcggggccgttaaggctccgccgcgaaagggactgcgtgggttcagacagcccccgggggacgccactcggttccggccagaatcgggccgtcttacaacttaccatttatgctcccacaatagatccgctccgctcatccagcccgcttgggtaccgtgcgcagcggcggccggggtgtgtcaagcgacagagggtcggccgagcaggcgcagccccggcgcaggttacgccatggcggccgctgattacaaggatgac

(19) United States
(12) Patent Application Publication
(10) Pub. No.: US 2026/0031187 A1
(43) Pub. Date: Jan. 29, 2026

(54) SYSTEMS AND METHODS FOR ENGINEERING CELL-TYPE SPECIFICITY IN MRNA

(71) Applicant: THE REGENTS OF THE UNIVERSITY OF CALIFORNIA, Oakland, CA (US)

(72) Inventors: Stephen Floor, San Francisco, CA (US); Yizhu Lin, San Francisco, CA (US)

(21) Appl. No.: 18/997,717

(22) PCT Filed: Jul. 21, 2023
(86) PCT No.: PCT/US2023/070769

§ 371 Date: Jan. 22, 2025

Related U.S. Application Data
(60) Provisional application No. 63/391,579, filed on Jul. 22, 2022.

(51) Int. Cl. G16B 35/10; C12N 15/10; G16B 40/20

(52) U.S. Cl. CPC G16B 35/10; C12N 15/1086; G16B 40/20

(57) ABSTRACT

Systems and methods for determining an effect regulatory untranslated RNA elements are provided. A plurality of RNA untranslated region (UTR) sequences are designed, subject to a requirement that each UTR RNA sequence includes one or more RNA regulatory elements in a plurality of RNA regulatory elements. The plurality of UTR RNA sequences samples a plurality of different spacings between each RNA regulatory element and a start or stop codon of a mRNA payload. The RNA UTR sequences are synthesized and cloned upstream or downstream of a mRNA payload to generate reporter constructs. The translation of each reporter construct is measured in a reporter cell type. These translation measurements, together with the sequences of the RNA UTR sequences, is used to train a model so that the model provides a quantitative translation estimate for a given test RNA UTR sequence whose sequence is inputted into the trained model.

### CROSS-REFERENCE TO RELATED APPLICATIONS

This application claims priority to U.S. Provisional Patent Application No. 63/391,579, filed on Jul. 22, 2022, which is hereby incorporated by reference in its entirety.

### TECHNICAL FIELD

The disclosure relates generally to determining an effect regulatory untranslated RNA elements have on protein synthesis.

### BACKGROUND

Synthetic messenger RNA (mRNA) therapeutics are poised to revolutionize medicine. The rapid deployment of mRNA-based vaccines against SARS-CoV-2 spike protein and variants demonstrates the power of mRNA therapeutics. Synthetic mRNAs have a major advantage over small molecules and antibodies: once the rules of protein production are defined, they are generalizable to any indication that can be treated by introduced gene expression. However, the potential of mRNA therapeutics is restricted by the limited understanding of how protein synthesis is controlled in human cells. Furthermore, selective delivery of mRNA therapeutics remains a challenge but cell-type specificity could be encoded in the sequence of the mRNA itself. See Blair et al., 2017, “Widespread Translational Remodeling during Human Neuronal Differentiation,” Cell Rep 21, 2005-2016; and Floor and Doudna, 2016, “Tunable protein synthesis by transcript isoforms in human cells,” eLife 5, e10921.

### SUMMARY

The present disclosure addresses the problems identified in the background. The systems and methods of the present disclosure makes use of developments in oligonucleotide synthesis, high-throughput sequencing, measurements of per-mRNA translation and machine learning to define the rules that regulate protein production in human cell types. The disclosed systems and methods have the practical application of advancing mRNA-based therapeutics for numerous diseases and pathogens, including many rare diseases with unmet medical need.

Methods for determining an effect one or more regulatory untranslated RNA elements have on protein synthesis are provided.

In accordance with the present disclosure there is designed, using a first computer system having one or more first processors, and a first memory storing one or more first programs for execution by the one or more first processors, a plurality of RNA untranslated region (UTR) sequences. Each RNA UTR sequence in the plurality of UTR RNA sequences has a length of at least 20 nucleotides. The designing is subjected to a first constraint that each UTR RNA sequence in the plurality of UTR RNA sequences includes one or more RNA regulatory elements, other than a start or stop codon, selected from a plurality of RNA regulatory elements. The designing is subjected to a second constraint that the plurality of UTR RNA sequences collectively samples a plurality of different spacings between each respective RNA regulatory element in the plurality of RNA regulatory elements and a start or stop codon of an mRNA payload gene. In some embodiments mRNA payload is a gene. In some embodiments, the mRNA payload is a reporter gene. In some embodiments, the mRNA payload is a therapeutic gene. In some embodiments the mRNA payload is gene that has a specific function (e.g., Cas9, spike protein, etc.).

In some embodiments, each RNA UTR sequence in the plurality of UTR RNA sequences has a length of at least 25 nucleotides, at least 50 nucleotides, at least 100 nucleotides, at least 150 nucleotides, at least 225 nucleotides, at least 250 nucleotides, at least 275 nucleotides, at least 300 nucleotides, at least 325 nucleotides, at least 350 nucleotides, at least 375 nucleotides, or at least 400 nucleotides.

In some embodiments, each RNA UTR sequence in the plurality of UTR RNA sequences has a length of between 20 nucleotides and 1000 nucleotides, between 225 nucleotides and 950 nucleotides, between 250 nucleotides and 900 nucleotides, or between 275 nucleotides and 850 nucleotides.

In some embodiments, an RNA regulatory element in the plurality of regulatory elements is an RNA-protein binding site having a size of between six and eight nucleotides.

In some embodiments, the plurality of RNA UTR sequences is at least 10,000 RNA UTR sequences, at least 100,000 RNA UTR sequences, at least 500,000 RNA UTR sequences, at least 1×10⁶ RNA UTR sequences, or at least 5×10⁶ RNA UTR sequences.

In some embodiments an RNA regulatory element in the plurality of regulatory elements is a first RNA structure comprising a 5′ portion of at least three nucleotides and a 3′ portion of at least three nucleotides, where the 5′ portion is complementary to the 3′ portion. In some such embodiments, the plurality of UTR RNA sequences collectively samples a plurality of different spacings between the first RNA structure and a start codon of the mRNA payload that is within the range of zero to 120 nucleotides.

In some embodiments the plurality of UTR RNA sequences collectively samples at least 3, at least 4, at least 5, at least 6, at least 7, at least 8, at least 9, or at least 10 different spacings between each respective RNA regulatory element in the plurality of RNA regulatory elements and a start or stop codon of the mRNA payload (e.g., gene).

Once designed, the plurality of RNA UTR sequences are synthesized.

Then, the plurality of synthesized RNA UTR sequences are cloned upstream or downstream of an RNA sequence encoding the mRNA payload, thereby generating a plurality of reporter constructs. Each reporter construct in the plurality of reporter construct comprises an RNA UTR sequence, from among the plurality of RNA UTR sequences, and the mRNA payload. In some embodiments, this cloning clones the plurality of RNA UTR sequences upstream of the RNA sequence encoding the mRNA payload. In other embodiments, this cloning clones the plurality of RNA UTR sequences downstream of the RNA sequence encoding the mRNA payload.

Translation of each reporter construct in the plurality of reporter constructs is measured in a reporter cell type, thereby determining a corresponding first quantitative translation label for each RNA UTR sequence in the plurality of RNA UTR sequences.

In some embodiments, this measuring involves transfecting the reporter cell type with the plurality of reporter constructs under conditions that transfect a single reporter construct in the plurality of reporter constructs into each cell of the reporter cell type prior to the measuring D). Then, fluorescence activated cell sorting (FACS) is used to measure translation of each reporter construct in the plurality of reporter constructs in a reporter cell type.

In some embodiments, the corresponding first quantitative translation label for a first RNA UTR sequence in the plurality of RNA UTR sequences is a distribution of the corresponding number of ribosomes attached to instances of the first RNA UTR sequence in the reporter cell type. In some such embodiments, a dynamic range of the corresponding first quantitative translation label is between zero ribosomes and ten ribosomes. Thus, in some such embodiments the distribution of the corresponding number of ribosomes attached to instances of the first RNA UTR sequence in the reporter cell type comprises: a count of a number of the first RNA UTR sequences in the reporter cell type that are attached to no ribosomes, a count of a number of the first RNA UTR sequences in the reporter cell type that are attached to one ribosome, a count of a number of the first RNA UTR sequences in the reporter cell type that are attached to two ribosomes, a count of a number of the first RNA UTR sequences in the reporter cell type that are attached to three ribosomes, a count of a number of the first RNA UTR sequences in the reporter cell type that are attached to four ribosomes, a count of a number of the first RNA UTR sequences in the reporter cell type that are attached to five ribosomes, a count of a number of the first RNA UTR sequences in the reporter cell type that are attached to six ribosomes, a count of a number of the first RNA UTR sequences in the reporter cell type that are attached to seven ribosomes, a count of a number of the first RNA UTR sequences in the reporter cell type that are attached to eight ribosomes, a count of a number of the first RNA UTR sequences in the reporter cell type that are attached to nine ribosomes, and a count of a number of the first RNA UTR sequences in the reporter cell type that are attached to ten ribosomes.

Using a second computer system having one or more second processors, and second memory storing one or more second programs for execution by the one or more second processors, an untrained model is trained using at least each sequence and corresponding first quantitative translation label of each RNA UTR sequence in the plurality of RNA UTR sequences thereby producing a trained model configured to provide a quantitative translation label upon input of a test RNA UTR sequence into the trained model.

In some such embodiments, the first computer system and the second computer system are the same. In other embodiments the first computer system and the second computer system are different.

In some embodiments, the trained model comprises a plurality of parameters, and the training sets a value of each parameter in the plurality of parameters. In some such embodiments, at least a subset of the plurality of UTR RNA sequences has two or more RNA regulatory elements selected from the plurality of RNA regulatory elements. The value of each parameter in at least a subset of parameters in the plurality of parameters is at least partially determined during the training E) by a second-order interaction term between a first RNA regulatory element and a second RNA regulatory element.

In some embodiments, the trained model comprises 10 or more parameters, 100 or more parameters, 1000 or more parameters, 10,000 or more parameters, 100,000 or more parameters or 1×10⁶ or more parameters.

In some embodiments, the trained model is a random forest regression model, a convolutional neural network model, a long short-term memory (LSTM) network, a logistic regression model, a neural network model, a support vector machine model, a Naive Bayes model, a nearest neighbor model, a boosted trees model, a random forest model, a decision tree model, a multinomial logistic regression model, a linear model, or a linear regression model. In some embodiments, the model is a regressor.

### BRIEF DESCRIPTION OF THE DRAWINGS

FIG. 1 illustrates a typical mammalian messenger RNA. The coding sequence in the dashed box encodes a polypeptide gene product, while the untranslated regions on both sides determine protein expression level, context-dependent protein synthesis, mRNA stability, mRNA localization, and other properties. Signaling pathways such as mTOR can regulate translation through the m7G cap, and various other regulatory elements are indicated.

FIG. 2 illustrates in vitro translation of RNA molecules with variable regulatory elements in accordance with the prior art. Changing the distance between an upstream start codon with weak sequence context and a downstream RNA secondary structure (n₁) results in changes to the probability of initiating at the upstream start codon.

FIG. 3 illustrates predicative quality of a model in accordance with an embodiment of the present disclosure as a function of the number of convolutional filters per layer of the model.

FIG. 4 illustrates how a designed library of one million mRNA 5′ untranslated regions with systematically perturbed regulatory elements such as protein binding sites, upstream start codons, and RNA secondary structures is used in accordance with an embodiment of the present disclosure. The regulatory element library is cloned upstream of a GFP reporter and in vitro transcribed and transfected into cells or transduced into cells. Measurements of protein expression through cell sorting or polysome profiles enable per-cell and per-mRNA analyses. Data are fed into simple random forest regression models and a convolutional neural network or long short-term memory (LSTM) network to model the impact of individual regulatory elements as well as second-order interaction terms between combinations of regulatory elements. This predictive model can be used with other cell types and conditions to understand how the learned rules are similar and different in different contexts.

FIG. 5 illustrates sample python code for generating a plurality of RNA untranslated region (UTR) sequences, in which it is understood that each “T” will in fact be a “U” in the RNA untranslated region (UTR) sequences, in accordance with an embodiment of the present disclosure.

FIG. 6 illustrates a computer system for determining an effect one or more regulatory untranslated RNA elements have on protein synthesis in accordance with an embodiment of the present disclosure.

FIGS. 7A, 7B, 7C, 7D, and 7E illustrate methods for determining an effect one or more regulatory untranslated RNA elements have on protein synthesis, in which optional method elements are indicated by dashed boxes, in accordance with an embodiment of the present disclosure.

FIG. 8 illustrates example data of highly and lowly translated mRNA untranslated regions.

FIG. 9 illustrates translation level of an entire library in accordance with an embodiment of the present disclosure.

FIG. 10 illustrates machine learning of determinants of translation level.

FIG. 11 illustrates features importance for translation level determined from machine learning.

### DETAILED DESCRIPTION

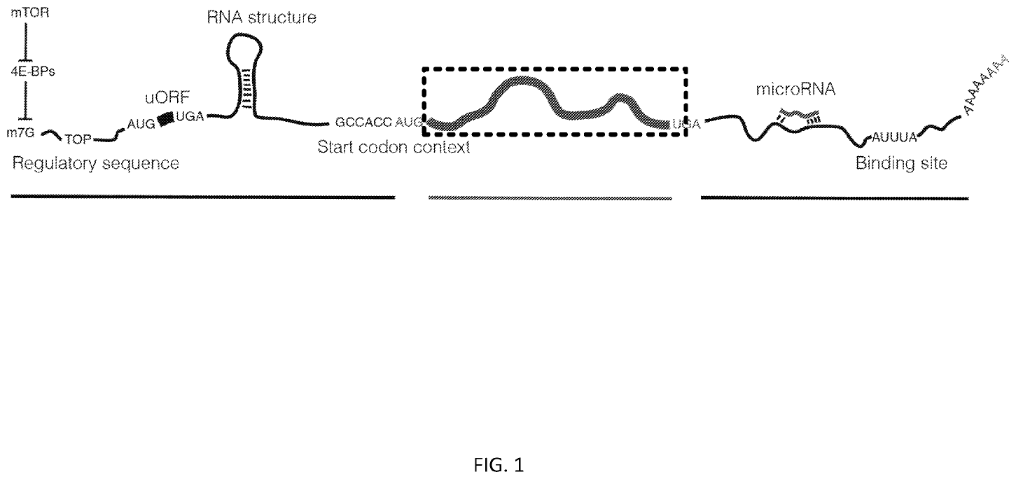

Human mRNAs contain a coding sequence that specifies the protein to produce, which is bracketed by regulatory untranslated regions (UTRs; FIG. 1). The UTRs of mRNA contain diverse regulatory elements that specify protein synthesis potential, subcellular localization, and mRNA stability. See Hinnebusch et al, 2016, “Translational control by 5′-untranslated regions of eukaryotic mRNAs,” Science 352, 1413-1416, which is hereby incorporated by reference. These regulatory elements are decoded by the translation machinery and RNA-binding proteins to make decisions about when, where, and how much protein to synthesize from an mRNA. Individual regulatory elements, such as intramolecular RNA structures, RNA sequences that are bound by specific proteins, the nucleotide context around start codons, upstream start codons that occur before the main start codon, microRNAs, RNA modifications, and more have been identified to date.

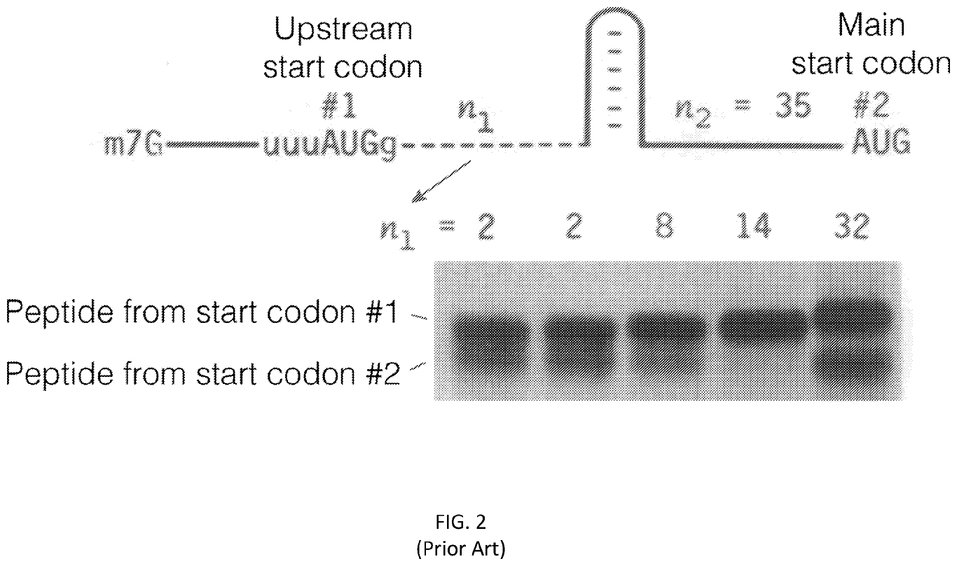

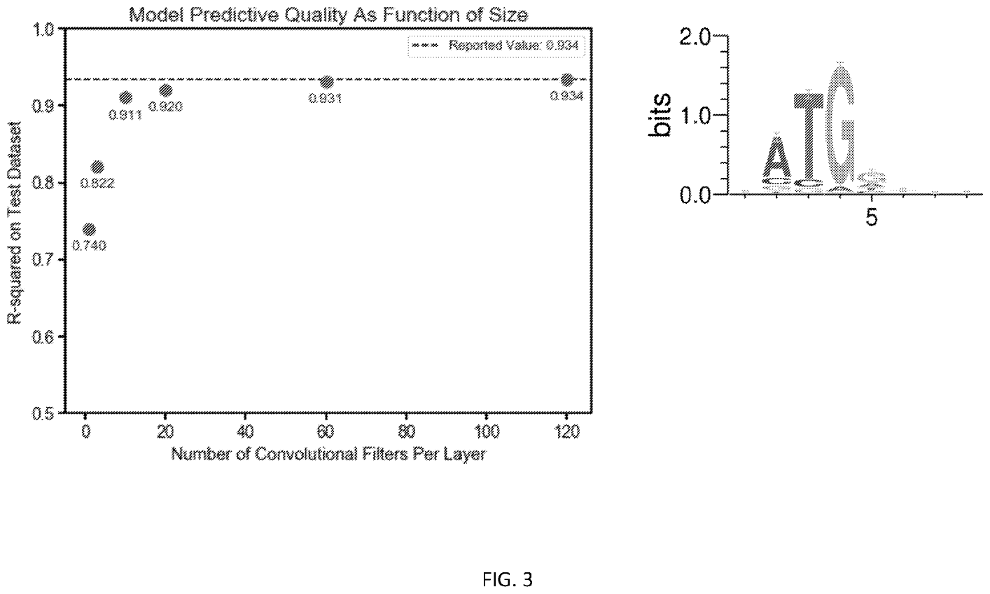

Despite extensive research into protein synthesis, there are large gaps in understanding that impact basic biology and the deployment of mRNA therapeutics. For example, regulatory elements are not all independent and can interact with each other, generating second-order effects. It is known that the distance between an RNA structure and an upstream start codon can change the probability of initiating translation. See, for instance, FIG. 2 modified from Kozak, 1990, “Downstream secondary structure facilitates recognition of initiator codons by eukaryotic ribosomes,” Proc. Natl. Acad. Sci. U.S.A. 87, 8301-8305, which is hereby incorporated by reference. Moreover, the set of factors that interact with RNA regulatory elements can change between cell states and cell types, leading to cell-type-specific rules. See Blair et al., 2017, “Widespread Translational Remodeling during Human Neuronal Differentiation,” Cell Rep 21, pp. 2005-2016, which is hereby incorporated by reference. However, both these effects are primarily understood in a qualitative manner. This is in part because most experiments rely on the natural diversity of human RNA sequences, small sequence perturbations, or random RNA sequences—none which efficiently sample the large sequence space of all possible RNA regulatory elements.

In present disclosure, synthetic libraries of mRNAs with systematically perturbed regulatory sequences are designed and machine learning is used to identify the rules that specify protein output. Following initial validation, protein output is measured from the synthesized library along human neuronal differentiation to identify cell-type-specific regulatory modules. Elucidation of these rules further synthetic biology—specifying protein production levels and cell-type specificity by designed mRNA sequences.

Unless defined otherwise, all technical and scientific terms used herein have the same meaning as is commonly understood by one of skill in the art to which this disclosure belongs. All patents and publications referred to herein are incorporated by reference in their entireties.

### Definitions

The term “in vivo” refers to an event that takes place in a subject's body. In some embodiments the subject is a human, an animal, or an organism (e.g., bacteria).

The term “in vitro” refers to an event that takes places outside of a subject's body. In vitro assays encompass cell-based assays in which cells alive or dead are employed and may also encompass a cell-free assay in which no intact cells are employed.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, or from 0% to 10%, or from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

As used interchangeably herein, the term “classifier” or “model” refers to a machine learning model or algorithm.

In some embodiments, a classifier is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a classifier is a multinomial classifier algorithm. In some embodiments, a classifier is a 2-stage stochastic gradient descent (SGD) model. In some embodiments, a classifier is a deep neural network (e.g., a deep-and-wide sample-level classifier).

Neuralnetworks. In some embodiments, the classifier is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in accordance with the present disclosure. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used in accordance with the present disclosure.

For instance, a deep neural network classifier comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network classifier. In some embodiments, at least 100 parameters, at least 1000 parameters, at least 2000 parameters or at least 5000 parameters are associated with the deep neural network classifier. As such, deep neural network classifiers require a computer to be used because they cannot be mentally solved. In other words, given an input to the classifier, the classifier output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as classifiers are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as classifiers are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as classifiers are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the classifier is a support vector machine (SVM). SVM algorithms suitable for use as classifiers are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM classifier requires a computer to calculate because it cannot be mentally solved.

Naive Bayes algorithms. In some embodiments, the classifier is a Naive Bayes algorithm. Naive Bayes classifiers suitable for use as classifiers are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes classifier is any classifier in a family of “probabilistic classifiers” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a classifier is a nearest neighbor algorithm. Nearest neighbor classifiers can be memory-based and include no classifier to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. In some embodiments, Euclidean distance in feature space is used to determine distance as d₍ᵢ₎=∥x₍ᵢ₎−x₍₀₎∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor classifier is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor classifier is such that a computer is used to solve the classifier for a given input because it cannot be mentally performed.

Random forest, decision tree, and boosted tree algorithms. In some embodiments, the classifier is a decision tree. Decision trees suitable for use as classifiers are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree classifier includes at least 10, at least 20, at least 50, or at least 100 parameters (e.g., weights and/or decisions) and requires a computer to calculate because it cannot be mentally solved.

Regression. In some embodiments, the classifier uses a regression algorithm. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the classifier. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the classifier makes use of a regression model disclosed in Hastie et al., 2001, The Elements of Statistical Learning, Springer-Verlag, New York. In some embodiments, the logistic regression classifier includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the classifier (linear classifier) in some embodiments of the present disclosure.

Mixture model and Hidden Markov model. In some embodiments, the classifier is a mixture model, such as that described in McLachlan et al., Bioinformatics 18(3):413-422, 2002. In some embodiments, in particular, those embodiments including a temporal component, the classifier is a hidden Markov model such as described by Schliep et al., 2003, Bioinformatics 19(1):i255-i263.

Clustering. In some embodiments, the classifier is an unsupervised clustering model. In some embodiments, the classifier is a supervised clustering model. Clustering algorithms suitable for use as classifiers are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of classifiers and boosting. In some embodiments, an ensemble (two or more) of classifiers is used. In some embodiments, a boosting technique such as AdaBoost is used in conjunction with many other types of learning algorithms to improve the performance of the classifier. In this approach, the output of any of the classifiers disclosed herein, or their equivalents, is combined into a weighted sum that represents the final output of the boosted classifier. In some embodiments, the plurality of outputs from the classifiers is combined using any measure of central tendency known in the art, including but not limited to a mean, median, mode, a weighted mean, weighted median, weighted mode, etc. In some embodiments, the plurality of outputs is combined using a voting method. In some embodiments, a respective classifier in the ensemble of classifiers is weighted or unweighted.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×106, n≥5×106, or n≥1×107. As such, the algorithms, models, regressors, and/or classifiers of the present disclosure cannot be mentally performed. In some embodiments n is between 10,000 and 1×107, between 100,000 and 5×106, or between 500,000 and 1×106. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “untrained model” (e.g., “untrained classifier” and/or “untrained neural network”) refers to a machine learning model or algorithm, such as a classifier or a neural network, that has not been trained on a target dataset. In some embodiments, “training a model” (e.g., “training a neural network”) refers to the process of training an untrained or partially trained model (e.g., “an untrained or partially trained neural network”). Moreover, it will be appreciated that the term “untrained model” does not exclude the possibility that transfer learning techniques are used in such training of the untrained or partially trained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained classifier described above is provided with additional data over and beyond that of the primary training dataset. Typically, this additional data is in the form of parameters (e.g., coefficients, weights, and/or hyperparameters) that were learned from another, auxiliary training dataset. Moreover, while a description of a single auxiliary training dataset has been disclosed, it will be appreciated that there is no limit on the number of auxiliary training datasets that may be used to complement the primary training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning may be used in such embodiments. For instance, consider the case where there is a first auxiliary training dataset and a second auxiliary training dataset in addition to the primary training dataset. The parameters learned from the first auxiliary training dataset (by application of a first classifier to the first auxiliary training dataset) may be applied to the second auxiliary training dataset using transfer learning techniques (e.g., a second classifier that is the same or different from the first classifier), which in turn may result in a trained intermediate classifier whose parameters are then applied to the primary training dataset and this, in conjunction with the primary training dataset itself, is applied to the untrained classifier. Alternatively, a first set of parameters learned from the first auxiliary training dataset (by application of a first classifier to the first auxiliary training dataset) and a second set of parameters learned from the second auxiliary training dataset (by application of a second classifier that is the same or different from the first classifier to the second auxiliary training dataset) may each individually be applied to a separate instance of the primary training dataset (e.g., by separate independent matrix multiplications) and both such applications of the parameters to separate instances of the primary training dataset in conjunction with the primary training dataset itself (or some reduced form of the primary training dataset such as principal components or regression coefficients learned from the primary training set) may then be applied to the untrained classifier in order to train the untrained classifier.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All embodiments of the invention can, in the alternative, be more specifically defined by any of the transitional terms “comprising,” “consisting essentially of,” and “consisting of.”

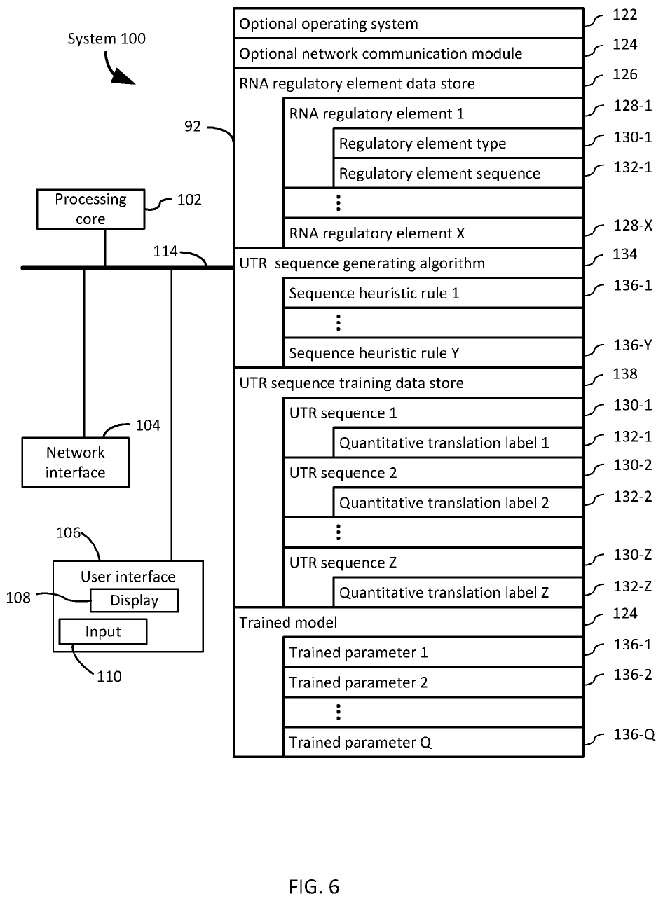

FIG. 6 illustrates a computer system 100 for determining an effect regulatory untranslated RNA elements have on protein synthesis. For instance, it can be used to provide an estimate of an ability of a particular untranslated RNA element or set of untranslated RNA elements upstream or downstream of a coding region in mRNA to affect the ability of the coding region to be translated.

Referring to FIG. 6, in typical embodiments, computer system 100 comprises one or more computers. For purposes of illustration in FIG. 6, the computer system 100 is represented as a single computer that includes all of the functionality of the disclosed computer system 100. However, the present disclosure is not so limited. The functionality of the computer system 100 may be spread across any number of networked computers and/or reside on each of several networked computers and/or virtual machines. One of skill in the art will appreciate that a wide array of different computer topologies are possible for the computer system 100 and all such topologies are within the scope of the present disclosure.

Turning to FIG. 6 with the foregoing in mind, the computer system 100 comprises one or more processing units (CPUs) 102, a network or other communications interface 104, a user interface 106 (e.g., including an optional display 108 and optional keyboard 110 or other form of input device), a memory 92 (e.g., random access memory, persistent memory, or combination thereof), and one or more communication busses 114 for interconnecting the aforementioned components. To the extent that components of memory 92 are not persistent, data in memory 92 can be seamlessly shared with non-volatile memory (not shown) or portions of memory 92 that are non-volatile/persistent using known computing techniques such as caching. Memory 92 can include mass storage that is remotely located with respect to the central processing unit(s) 102. In other words, some data stored in memory 92 may in fact be hosted on computers that are external to computer system 100 but that can be electronically accessed by the computer system 100 over an Internet, intranet, or other form of network or electronic cable using network interface 104. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

The memory 92 of the computer system 100 stores:
an optional operating system 122 that includes procedures for handling various basic system services;
an optional network communication module for accessing data from or providing data or results to a computer system external to system 100 over a computer network;
an RNA regulatory element data store 126 comprises details of a plurality of RNA regulatory elements 128 to be analyzed including, for each respective regulatory element, the regulatory element type 130 and sequence 132;
a UTR sequence generating algorithm 134 for using the RNA regulatory elements 128 in the RNA regulatory element data store 126 to generate UTR sequences in accordance with a plurality of sequence heuristic rules 136;
a UTR sequence training data store 138 that contains the UTR sequences 130 generated in accordance with the UTR sequence generating algorithm 134, the UTR sequence training data store 138 including for each respective UTR sequence 130 a corresponding measured quantitative translation label 132; and
a trained model 124, trained on the data of the UTR sequence training data store 138, the trained model therefore comprising a plurality of trained parameter 136, the trained model configured to provide a quantitative translation label upon input of a test RNA UTR sequence into the trained model.

In some implementations, one or more of the above identified data elements or modules of the computer system 100 are stored in one or more of the previously mentioned memory devices, and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 92 optionally stores a subset of the modules and data structures identified above. Furthermore, in some embodiments the memory 92 stores additional modules and data structures not described above.

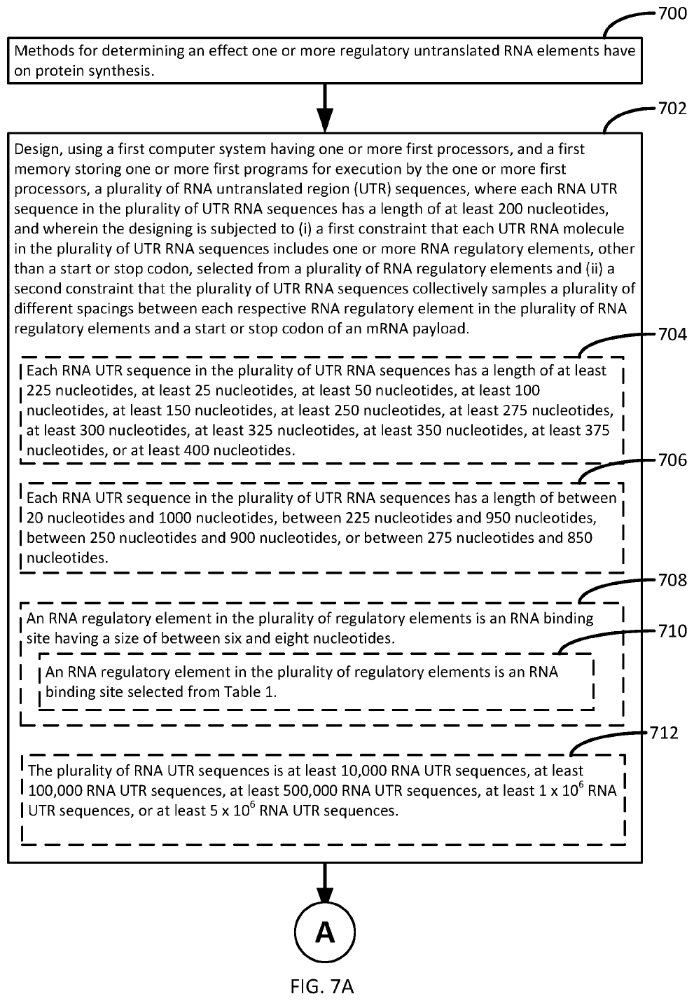

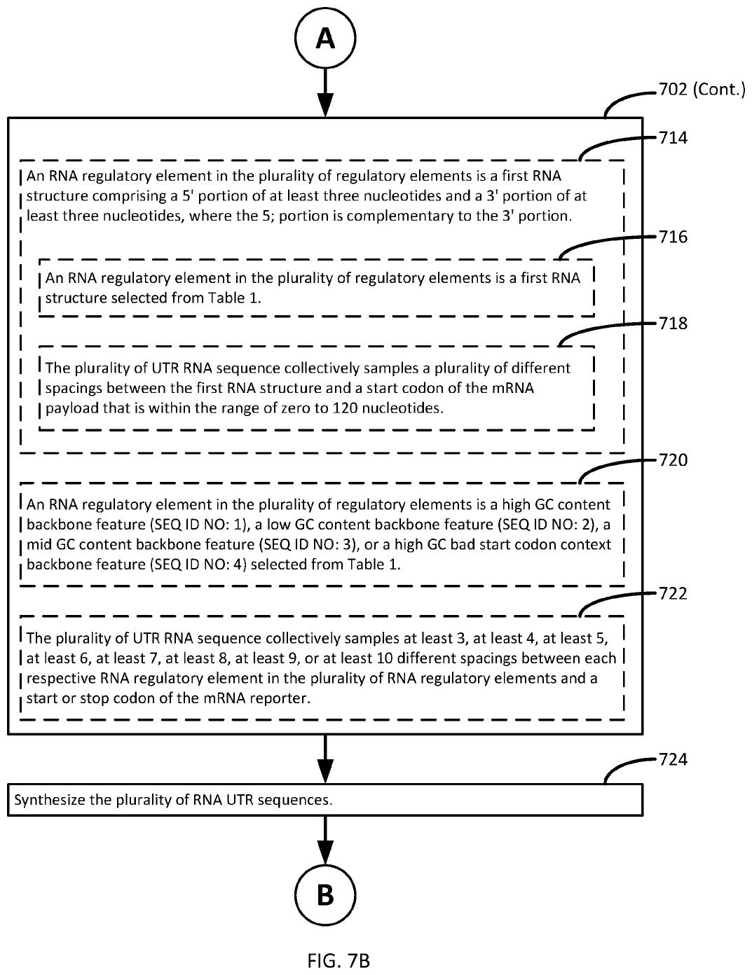

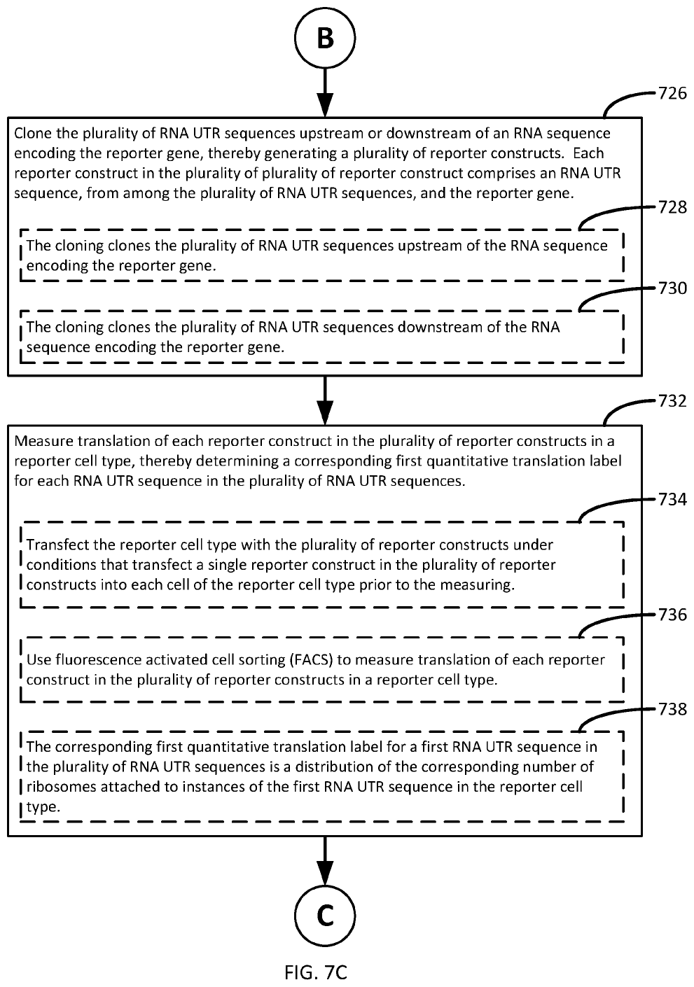

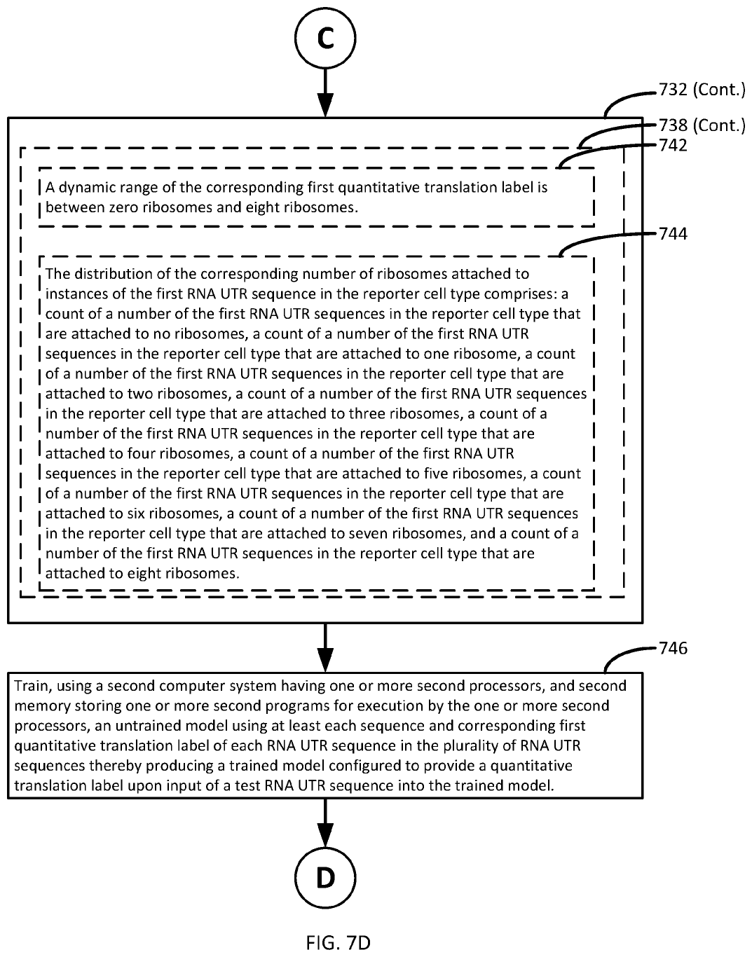

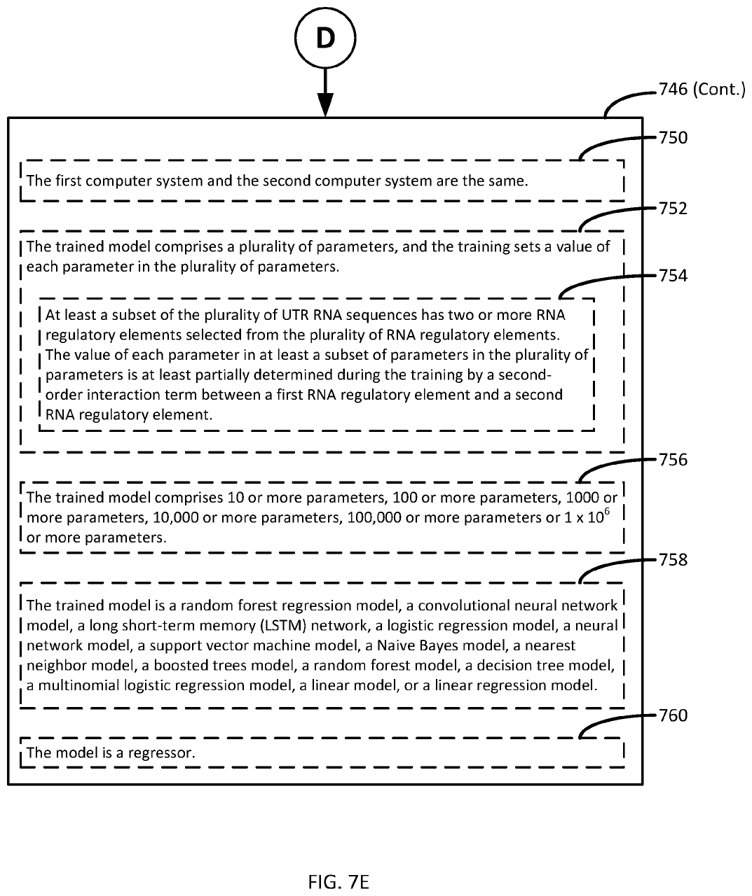

Now that a system for characterizing an interaction between a test compound and a target polymer has been disclosed, methods for performing such characterization is detailed with reference to FIG. 7 and discussed below.

Block 700. In accordance with block 700 of FIG. 7A, methods for determining an effect one or more regulatory untranslated RNA elements have on protein synthesis are provided

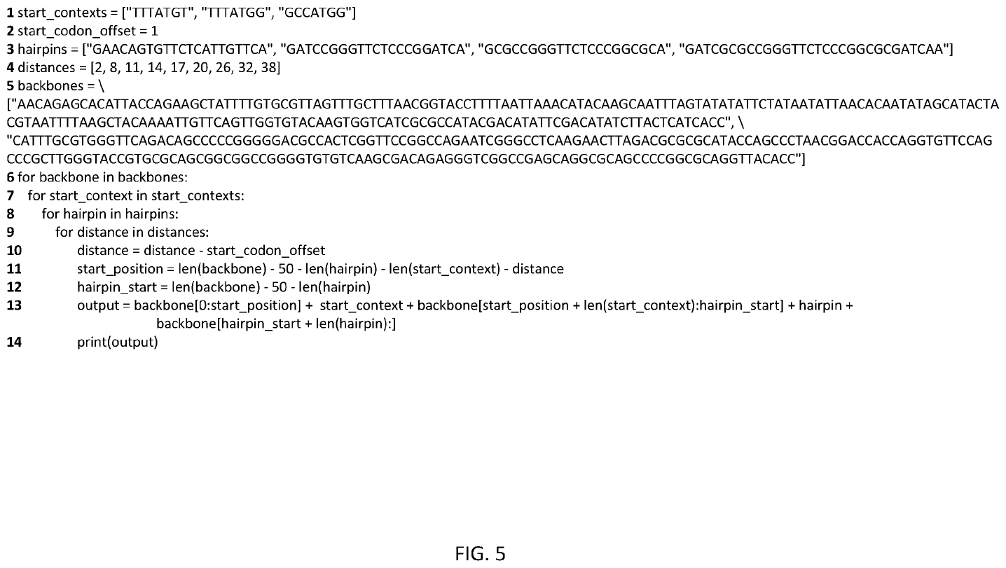

Blocks 702-722. In accordance with block 702 of FIG. 7A, in accordance with the present disclosure there is designed, using a first computer system having one or more first processors, and a first memory storing one or more first programs for execution by the one or more first processors, a plurality of RNA untranslated region (UTR) sequences. Each RNA UTR sequence in the plurality of UTR RNA molecules has a length of at least 20 nucleotides. The designing is subject to a first constraint that each UTR RNA sequence in the plurality of UTR RNA sequences includes one or more RNA regulatory elements, other than a start or stop codon, selected from a plurality of RNA regulatory elements. Example RNA regulatory elements are disclosed in Table 1 below and further discussion of RNA regulatory elements is provided Example 1 below. The designing is subject to a second constraint that the plurality of UTR RNA sequences collectively samples a plurality of different spacings between each respective RNA regulatory element in the plurality of RNA regulatory elements and a start or stop codon of an mRNA payload. An example of such spacings is illustrated in Example 1 below in conjunction with the code illustrated in FIG. 5.

In accordance with block 704 of FIG. 7A, in some embodiments, each RNA UTR sequence in the plurality of UTR RNA sequences has a length of at least 25 nucleotides, at least 50 nucleotides, at least 100 nucleotides, at least 150 nucleotides, at least 225 nucleotides, at least 250 nucleotides, at least 275 nucleotides, at least 300 nucleotides, at least 325 nucleotides, at least 350 nucleotides, at least 375 nucleotides, or at least 400 nucleotides.

In accordance with block 706 of FIG. 7A, in some embodiments, each RNA UTR sequence in the plurality of UTR RNA sequences has a length of between 20 nucleotides and 1000 nucleotides, between 225 nucleotides and 950 nucleotides, between 250 nucleotides and 900 nucleotides, or between 275 nucleotides and 850 nucleotides.

In accordance with block 708 of FIG. 7A, in some embodiments, an RNA regulatory element in the plurality of regulatory elements is an RNA binding site having a size of between six and eight nucleotides. Examples of such binding site regulatory elements in accordance with the present disclosure is provided in Table 1 below (Block 710).

TABLE 1

| Feature type | Sequence |
|---|---|
| high GC content backbone | CATTUGCGUGGGUUCAGACAGCCCCCGGGGG |
| | ACGCCACUCGGUUCCGGCCAGAAUCGGGCC |
| | UCAAGAACUUAGACGCGCGCAUACCAGCCC |
| | UAACGGACCACCAGGUGUUCCAGCCCGCUU |
| | GGGUACCGUGCGCAGCGGCGGCCGGGGUGU |
| | GUCAAGCGACAGAGGGUCGGCCGAGCAGGC |
| | GCAGCCCCGGCGCAGGUUACGCC (SEQ ID |
| | NO: 1) |
| low GC content backbone | AACAGAGCACAUUACCAGAAGCUAUUUUGU |
| | GCGUUAGUUUGCUUUAACGGUACCUUUUAA |
| | UUAAACAUACAAGCAAUUUAGUAUAUAUUC |
| | UAUAAUAUUAACACAAUAUAGCAUACUACG |
| | UAAUUUUAAGCUACAAAAUUGUUCAGUUGG |
| | UGUACAAGUGGUCAUCGCGCCAUACGACAU |
| | AUUCGACAUAUCUUACUCAUCGCC (SEQ ID |
| | NO: 2) |
| mid GC content backbone | UUCCCGGGACCCGGUCGUGGGUUCGAGUCA |
| | CCUAAAGGAGCAAAAGCGUGUUAUUGUAGU |
| | UUCGAGGCACGGAACUCAAUACCAUUACGG |
| | UGAACCCCACGAACCUUCGACGUUUGGGCA |
| | UAUAUAUUCUCACGCAGAAAGUCAGGAUAG |
| | GACUUGCGGGUAUACAGUGCCAGCCGCUUC |
| | GCUCUACUUUAACUAACGAUCGCC (SEQ ID |
| | NO: 3) |
| high GC bad start codon context | CAUUUGCGUGGGUUCAGACAGCCCCCGGGG |
| backbone | GACGCCACUCGGUUCCGGCCAGAAUCGGGC |
| | CUCAAGAACUUAGACGCGCGCAUACCAGCC |
| | CUAACGGACCACCAGGUGUUCCAGCCCGCU |
| | UGGGUACCGUGCGCAGCGGCGGCCGGGGUG |
| | UGUCAAGCGACAGAGGGUCGGCCGAGCAGG |
| | CGCAGCCCCGGCGCAGGUUACUUU (SEQ ID |
| | NO: 4) |
| start codon | AUG |
| start codon | CUG |
| stop codon | UAA |
| stop codon | UAG |
| stop codon | UGA |
| RNA structure | UUGGGCCGUUAAGGCUCAA (SEQ ID NO: 5) |
| RNA structure | CGGCGGGGCCGUUAAGGCUCCGCCG (SEQ ID |
| | NO: 6) |
| RNA structure | GUCCCUUUCGCGGCGGGGCCGUUAAGGCUC |
| | CGCCGCGAAAGGGAC (SEQ ID NO: 7) |
| RNA structure | GAUCCGGGUUCUCCCGGAUC (SEQ ID NO: 8) |
| RNA structure | CGCGGCGGGGCCGUUAAGGCUCCGCCGCG |
| | (SEQ ID NO: 9) |
| RNA structure | GGGCUGCGGGCCUCCGCAGCCC (SEQ ID NO: |
| | 10) |
| RNA structure | GGGCCGUGGUGGAGCUUCCACCAUGGCCC |
| | (SEQ ID NO: 11) |
| RNA structure | GGGGCGCGUGGUGGCGGCUGCAGCCGCCAC |
| | CACGCGCCCC (SEQ ID NO: 12) |
| RNA structure | CGTCCAGATCTACATAGAGTACGTACGCGTA |
| | GATCTGACG (SEQ ID NO: 13) |
| RNA structure | CGCTGCGACGGCAGCG (SEQ ID NO: 14) |
| RNA structure | TGCGACGGCA (SEQ ID NO: 15) |
| RNA structure | CGGCTCCGCGACGGCGGAGCCG (SEQ ID NO: |
| | 16) |
| RNA structure | CTGCTACGCGACGGCGTAGCAG (SEQ ID NO: |
| | 17) |
| RNA structure | CAATTTCCATCTTGGAAATTG (SEQ ID NO: 18) |
| RNA structure | CAATTATCATCTTGGTAATTG (SEQ ID NO: 19) |
| RNA structure | CAATTTCCATCTTGGCAATTG (SEQ ID NO: 20) |
| RNA structure | CAATTCCCATCTTGGGAATTG (SEQ ID NO: 21) |
| RNA structure | GAATTAAATCTGTTTAATTC (SEQ ID NO: 22) |
| RNA structure | GAATTACCATCTTGGGACTTC (SEQ ID NO: 23) |
| RNA structure | CGGATCCGGCCGGATCCGGCCGGATCCG |
| | (SEQ ID NO: 24) |
| RNA structure | CCCAACCUGCCGGAUGCGGCAGGUUGGG |
| | (SEQ ID NO: 25) |
| RNA structure | CGGACCCGGCCGGATACGGCCGGGTCCG |
| | (SEQ ID NO: 26) |
| RNA structure | CGGGCGCGTGGTGGCGGCTGCAGCCGCCACC |
| | ACGCGCCCG (SEQ ID NO: 27) |
| Start codon context | GCCACC |
| Start codon context | GCCGCC |
| Start codon context | GUUACC |
| Start codon context | GUUGCC |
| Start codon context | GUUUCC |
| RNA binding protein binding site | AAUUA |
| RNA binding protein binding site | UUUUU |
| RNA binding protein binding site | UAUGU |
| RNA binding protein binding site | ACACA |
| RNA binding protein binding site | UUUUU |
| RNA binding protein binding site | AGUUA |
| RNA binding protein binding site | AUAUA |
| RNA binding protein binding site | GUUGC |
| RNA binding protein binding site | UUUUU |
| RNA binding protein binding site | GGGGG |
| RNA binding protein binding site | GGGGG |
| RNA binding protein binding site | UUUUU |
| RNA binding protein binding site | UAUAU |
| RNA binding protein binding site | GGGGG |
| RNA binding protein binding site | UAUAG |
| RNA binding protein binding site | GGGGG |
| RNA binding protein binding site | UUUUU |
| RNA binding protein binding site | UUUUU |
| RNA binding protein binding site | UAUUA |
| RNA binding protein binding site | UAAUU |
| RNA binding protein binding site | GGGGG |
| RNA binding protein binding site | GGGGG |
| RNA binding protein binding site | GCCCA |
| RNA binding protein binding site | ACACA |
| RNA binding protein binding site | AUACA |
| RNA binding protein binding site | ACACA |
| RNA binding protein binding site | GGGGG |
| RNA binding protein binding site | AUAAA |
| RNA binding protein binding site | AUAAA |
| RNA binding protein binding site | UGUAU |
| RNA binding protein binding site | CGCUU |
| RNA binding protein binding site | UAGUU |
| RNA binding protein binding site | UUCAU |
| RNA binding protein binding site | AAAAA |
| RNA binding protein binding site | AAAAA |
| RNA binding protein binding site | GCCCC |
| RNA binding protein binding site | CCCCC |
| RNA binding protein binding site | AUCCC |
| RNA binding protein binding site | AUAAG |
| RNA binding protein binding site | UUUCU |
| RNA binding protein binding site | UUUUU |
| RNA binding protein binding site | UGUAU |
| RNA binding protein binding site | UUUUU |
| RNA binding protein binding site | GCAUG |
| RNA binding protein binding site | GCAUG |
| RNA binding protein binding site | UUUUU |
| RNA binding protein binding site | ACCGG |
| RNA binding protein binding site | CCCCC |
| RNA binding protein binding site | GUGUG |
| RNA binding protein binding site | GGGGG |
| RNA binding protein binding site | GCGCG |
| RNA binding protein binding site | UACUU |
| RNA binding protein binding site | ACGCA |
| RNA binding protein binding site | AAUCA |
| RNA binding protein binding site | GCGCG |
| RNA binding protein binding site | CGUCC |
| RNA binding protein binding site | AUAUA |
| RNA binding protein binding site | UAUAU |
| RNA binding protein binding site | AUAUU |
| RNA binding protein binding site | UAACA |
| RNA binding protein binding site | UGUAA |
| RNA binding protein binding site | UGCAC |
| RNA binding protein binding site | AGCAG |
| RNA binding protein binding site | AGGGG |
| RNA binding protein binding site | AGCAG |
| RNA binding protein binding site | AGCAG |
| RNA binding protein binding site | GCAGC |
| RNA binding protein binding site | GCAGC |
| RNA binding protein binding site | AGGAG |
| RNA binding protein binding site | GGGGG |
| RNA binding protein binding site | GUAUG |
| RNA binding protein binding site | UUUUU |
| RNA binding protein binding site | GAAGA |
| RNA binding protein binding site | UUUUU |
| RNA binding protein binding site | UUAGU |
| RNA binding protein binding site | UUAAU |
| RNA binding protein binding site | UAUUU |
| RNA binding protein binding site | GGAUG |

In accordance with block 712 of FIG. 7A, in some embodiments the plurality of RNA UTR sequences is at least 10,000 RNA UTR sequences, at least 100,000 RNA UTR sequences, at least 500,000 RNA UTR sequences, at least 1×10⁶ RNA UTR sequences, or at least 5×10⁶ RNA UTR sequences.

In accordance with block 714 of FIG. 7B, in some embodiments, an RNA regulatory element in the plurality of regulatory elements is a first RNA structure comprising a 5′ portion of at least three nucleotides and a 3′ portion of at least three nucleotides, where the 5′ portion is complementary to the 3′ portion.

In accordance with block 716 of FIG. 7B, in some embodiments, an RNA regulatory element in the plurality of regulatory elements is a first RNA structure selected from Table 1.

In accordance with block 718 of FIG. 7B, in some embodiments, the plurality of UTR RNA sequences collectively samples a plurality of different spacings between the first RNA structure and a start codon of the mRNA payload that is within the range of zero to 120 nucleotides.

In accordance with block 720 of FIG. 7B, in some embodiments, an RNA regulatory element in the plurality of regulatory elements is a high GC content backbone feature (SEQ ID NO: 1), a low GC content backbone feature (SEQ ID NO: 2), a mid GC content backbone feature (SEQ ID NO: 3), or a high GC bad start codon context backbone feature (SEQ ID NO: 4) selected from Table 1.

In accordance with block 722 of FIG. 7B, in some embodiments, the plurality of UTR RNA sequences collectively samples at least 3, at least 4, at least 5, at least 6, at least 7, at least 8, at least 9, or at least 10 different spacings between each respective RNA regulatory element in the plurality of RNA regulatory elements and a start or stop codon of the mRNA payload.

Block 724. One the plurality of RNA UTR sequences have been designed they are synthesized as DNA in accordance with block 724 of FIG. 7B.

Block 726-730. The synthesized plurality of DNA encoding RNA UTR sequences are then cloned upstream or downstream of an DNA sequence encoding the mRNA payload, thereby generating a plurality of reporter constructs in accordance with block 726 of FIG. 7C. In some such embodiments, each reporter construct in the plurality of reporter construct comprises an sequence, from among the plurality of RNA UTR sequences, and the mRNA payload. In some such embodiments the cloning clones the plurality of RNA UTR sequences upstream of the RNA sequence encoding the mRNA payload (block 728). In other embodiments, the cloning clones the plurality of RNA UTR sequences downstream of the RNA sequence encoding the mRNA payload (block 728).

Blocks 732-744. In accordance with block 732 of FIG. 7C, the method continues with the measurement of the translation of each reporter construct in the plurality of reporter constructs in a reporter cell type, thereby determining a corresponding first quantitative translation label for each RNA UTR sequence in the plurality of RNA UTR sequences.

In accordance with block 734 in FIG. 7C, in some embodiments, the reporter cell type is transfected with the plurality of reporter constructs under conditions that transfect a single reporter construct in the plurality of reporter constructs into each cell of the reporter cell type prior to the measuring of block 732. In accordance with block 736, in some such embodiments, fluorescence activated cell sorting (FACS) is used to measure translation of each reporter construct in the plurality of reporter constructs in a reporter cell type.

In accordance with block 738 of FIG. 7C, in some embodiments the corresponding first quantitative translation label for a first RNA UTR sequence in the plurality of RNA UTR sequences is a distribution of the corresponding number of ribosomes attached to instances of the first RNA UTR sequence in the reporter cell type.

In accordance with block 742 of FIG. 7D, in some embodiments a dynamic range of the corresponding first quantitative translation label is between zero ribosomes and ten ribosomes. For instance, in some such embodiments in accordance with block 744, the distribution of the corresponding number of ribosomes attached to instances of the first RNA UTR sequence in the reporter cell type comprises: a count of a number of the first RNA UTR sequences in the reporter cell type that are attached to no ribosomes, a count of a number of the first RNA UTR sequences in the reporter cell type that are attached to one ribosome, a count of a number of the first RNA UTR sequences in the reporter cell type that are attached to two ribosomes, a count of a number of the first RNA UTR sequences in the reporter cell type that are attached to three ribosomes, a count of a number of the first RNA UTR sequences in the reporter cell type that are attached to four ribosomes, a count of a number of the first RNA UTR sequences in the reporter cell type that are attached to five ribosomes, a count of a number of the first RNA UTR sequences in the reporter cell type that are attached to six ribosomes, a count of a number of the first RNA UTR sequences in the reporter cell type that are attached to seven ribosomes, a count of a number of the first RNA UTR sequences in the reporter cell type that are attached to eight ribosomes, a count of a number of the first RNA UTR sequences in the reporter cell type that are attached to nine ribosomes, and a count of a number of the first RNA UTR sequences in the reporter cell type that are attached to ten ribosomes.

Blocks 746-760. In accordance with block 746 of FIG. 7D, at least each sequence and corresponding first quantitative translation label of each RNA UTR sequence in the plurality of RNA UTR sequences is used to train, at a second computer system having one or more second processors, and second memory storing one or more second programs for execution by the one or more second processors, an untrained model using thereby producing a trained model configured to provide a quantitative translation label upon input of a test RNA UTR sequence into the trained model.

In accordance with block 750 of FIG. 7E, in some embodiments the first computer system and the second computer system are the same.

In accordance with block 752 of FIG. 7E, in some embodiments, the trained model comprises a plurality of parameters, and the training sets a value of each parameter in the plurality of parameters.

In accordance with block 754 of FIG. 7E, in some embodiments at least a subset of the plurality of UTR RNA sequences has two or more RNA regulatory elements selected from the plurality of RNA regulatory elements. The value of each parameter in at least a subset of parameters in the plurality of parameters is at least partially determined during the training by a second-order interaction term between a first RNA regulatory element and a second RNA regulatory element.

In accordance with block 756 of FIG. 7E, in some embodiments the trained model comprises 10 or more parameters, 100 or more parameters, 1000 or more parameters, 10,000 or more parameters, 100,000 or more parameters or 1×10⁶ or more parameters.

In accordance with block 758 of FIG. 7E, in some embodiments the trained model is a random forest regression model, a convolutional neural network model, a long short-term memory (LSTM) network, a logistic regression model, a neural network model, a support vector machine model, a Naive Bayes model, a nearest neighbor model, a boosted trees model, a random forest model, a decision tree model, a multinomial logistic regression model, a linear model, or a linear regression model.

In accordance with block 760, in some embodiments the model is a regressor.

### Example—Building a Synthetic mRNA Library to Systematically Explore Regulatory Element Space

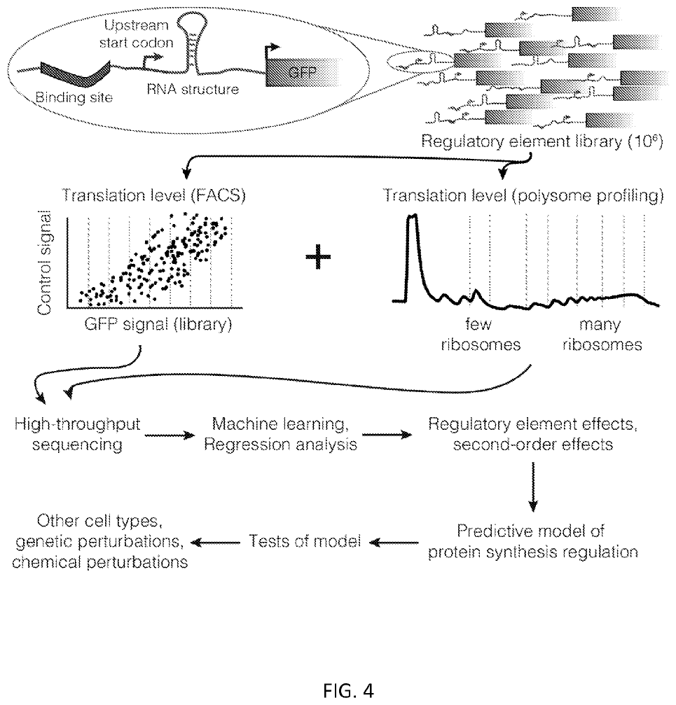

This example details a project summarized in FIG. 4 for elucidating the effects of regulatory elements on the control of mRNA transcription. An algorithm to generate a library of 300-nucleotide-long 5′ UTRs each containing defined combinations of regulatory elements was written to systematically explore RNA regulation. The length scale of 300 nucleotides is similar to the median human 5′ UTR length (See Leppek et al., 2018, “Functional 5′ UTR mRNA structures in eukaryotic translation regulation and how to find them,” Nat. Rev. Mol. Cell Biol. 19, pp. 158-174, which is hereby incorporated by reference). Use of this length scale allows for the sampling of physiologically relevant regulatory element configurations.

An example of code that is used to make such systematic UTRs is illustrated in FIG. 5. The code for creation of the library for this example is necessarily more complex because it samples more regulatory elements than our sampled in FIG. 5.

Line 1 of the sample code, written in Python, defines three different example start contexts, “TTTATGT”, “TTTATGG”, and “GCCATGG”.

Line 2 of the sample code defines a start codon offset. Line 3 of the code defines four different RNA structures that will be evaluated: “GAACAGTGTTCTCATTGTTCA” (SEQ ID NO: 28), “GATCCGGGTTCTCCCGGATCA” (SEQ ID NO: 29), “GCGCCGGGTTCTCCCGGCGCA” (SEQ ID NO: 30), and “GATCGCGCCGGGTTCTCCCGGCGCGATCAA” (SEQ ID NO: 31), where it is understood that each “T” will be a “U” when tested in vivo.

Line 4 of the code defines 9 different nucleotide offset distances to sample: 2, 8, 11, 14, 17, 20, 26, 32, and 38.

Next, line 5 of the sample code defines two different backbones:

(SEQ ID NO: 32)
“AACAGAGCACATTACCAGAAGCTATTTTGTGCGTTAGTTTGCTTTAACG
GTACCTTTTAATTAAACATACAAGCAATTTAGTATATATTCTATAATATT
AACACAATATAGCATACTACGTAATTTTAAGCTACAAAATTGTTCAGTTG
GTGTACAAGTGGTCATCGCGCCATACGACATATTCGACATATCTTACTCA
TCACC”
and
(SEQ ID NO: 33)
“CATTTGCGTGGGTTCAGACAGCCCCCGGGGGACGCCACTCGGTTCCGGC
CAGAATCGGGCCTCAAGAACTTAGACGCGCGCATACCAGCCCTAACGGAC
CACCAGGTGTTCCAGCCCGCTTGGGTACCGTGCGCAGCGGCGGCCGGGGT
GTGTCAAGCGACAGAGGGTCGGCCGAGCAGGCGCAGCCCCGGCGCAGGTT
ACACC”

Next, lines 6 through 14 generates a series of substitutions of each of the two backbones. For each respective backbone in the two backbones, for each respective start context in the three different start contexts, for each respective hairpin in the four different hairpins, for each respective offset distance in the 9 offset distances, the respective start context and the respective hairpin are substituted into different portions of the respective backbone. The respective start context is substituted into the backbone beginning at a position in the respective backbone defined by the length of the respective backbone—50 nucleotides—the length of the respective hairpin—the length of the start context—the length of the respective offset distance as defined by line 11 of the code. As such, line 11 of the code represents a form of sequence heuristic rule 136 and the code of FIG. 5 illustrates one embodiment of the UTR sequence generating algorithm depicted in FIG. 6. The respective hairpin is substituted into the backbone beginning at a position in the respective backbone defined by the length of the backbone—50 nucleotides—the length of the hairpin element. Thus, in this example, the start context is sampled in 9 different positions in each of the two different backbones whereas the hairpin always begins 50 nucleotides away from the 3′ end of the backbone. The script illustrated in FIG. 5 thus generates a total of 2 (backbones)×3 (start contexts)×4 (hairpins)×9 (sampling distances) or 216 sequences. The first 10 of these 216 sequences, for purposes of illustration, is provided in Table 2 below.

TABLE 2
Sample sequences generated by the code of FIG. 5

| Sequence | |
|---|---|
| No. | Sequence |
| 1 | CATTTGCGTGGGTTCAGACAGCCCCCGGGGGACGCCACTCGGTTCCGGCCAGAATC |
| | GGGCCTCAAGAACTTAGACGCGCGCATACCAGCCCTAACGGACCACCAGGTGTTCC |
| | AGCCCGCGCCATGGCCGTGCGGAACAGTGTTCTCATTGTTCAAAGCGACAGAGGGT |
| | CGGCCGAGCAGGCGCAGCCCCGGCGCAGGTTACACC (SEQ ID NO: 34) |
| 2 | CATTTGCGTGGGTTCAGACAGCCCCCGGGGGACGCCACTCGGTTCCGGCCAGAATC |
| | GGGCCTCAAGAACTTAGACGCGCGCATACCAGCCCTAACGGACCACCAGGTGTTCC |
| | AGCCGCCATGGGTACCGTGCGGAACAGTGTTCTCATTGTTCAAAGCGACAGAGGGT |
| | CGGCCGAGCAGGCGCAGCCCCGGCGCAGGTTACACC (SEQ ID NO: 35) |
| 3 | CATTTGCGTGGGTTCAGACAGCCCCCGGGGGACGCCACTCGGTTCCGGCCAGAATC |
| | GGGCCTCAAGAACTTAGACGCGCGCATACCAGCCCTAACGGACCACCAGGTGTTCC |
| | AGCCATGGTGGGTACCGTGCGGAACAGTGTTCTCATTGTTCAAAGCGACAGAGGGT |
| | CGGCCGAGCAGGCGCAGCCCCGGCGCAGGTTACACC (SEQ ID NO: 36) |
| 4 | CATTTGCGTGGGTTCAGACAGCCCCCGGGGGACGCCACTCGGTTCCGGCCAGAATC |
| | GGGCCTCAAGAACTTAGACGCGCGCATACCAGCCCTAACGGACCACCAGGTGTTGC |
| | CATGGGCTTGGGTACCGTGCGGAACAGTGTTCTCATTGTTCAAAGCGACAGAGGGT |
| | CGGCCGAGCAGGCGCAGCCCCGGCGCAGGTTACACC (SEQ ID NO: 37) |
| 5 | CATTTGCGTGGGTTCAGACAGCCCCCGGGGGACGCCACTCGGTTCCGGCCAGAATC |
| | GGGCCTCAAGAACTTAGACGCGCGCATACCAGCCCTAACGGACCACCAGGTGCCAT |
| | GGCCCGCTTGGGTACCGTGCGGAACAGTGTTCTCATTGTTCAAAGCGACAGAGGGT |
| | CGGCCGAGCAGGCGCAGCCCCGGCGCAGGTTACACC (SEQ ID NO: 38) |
| 6 | CATTTGCGTGGGTTCAGACAGCCCCCGGGGGACGCCACTCGGTTCCGGCCAGAATC |
| | GGGCCTCAAGAACTTAGACGCGCGCATACCAGCCCTAACGGACCAGCCATGGTTCC |
| | AGCCCGCTTGGGTACCGTGCGGAACAGTGTTCTCATTGTTCAAAGCGACAGAGGGT |
| | CGGCCGAGCAGGCGCAGCCCCGGCGCAGGTTACACC (SEQ ID NO: 39) |
| 7 | CATTTGCGTGGGTTCAGACAGCCCCCGGGGGACGCCACTCGGTTCCGGCCAGAATC |
| | GGGCCTCAAGAACTTAGACGCGCGCATACCAGCCCTAACGCCATGGCAGGTGTTCC |
| | AGCCCGCTTGGGTACCGTGCGGAACAGTGTTCTCATTGTTCAAAGCGACAGAGGGT |
| | CGGCCGAGCAGGCGCAGCCCCGGCGCAGGTTACACC (SEQ ID NO: 40) |
| 8 | CATTTGCGTGGGTTCAGACAGCCCCCGGGGGACGCCACTCGGTTCCGGCCAGAATC |
| | GGGCCTCAAGAACTTAGACGCGCGCATACCAGCGCCATGGGACCACCAGGTGTTCC |
| | AGCCCGCTTGGGTACCGTGCGGAACAGTGTTCTCATTGTTCAAAGCGACAGAGGGT |
| | CGGCCGAGCAGGCGCAGCCCCGGCGCAGGTTACACC (SEQ ID NO: 41) |
| 9 | CATTTGCGTGGGTTCAGACAGCCCCCGGGGGACGCCACTCGGTTCCGGCCAGAATC |
| | GGGCCTCAAGAACTTAGACGCGCGCATACCAGCCCTAACGGACCACCAGGTGTTCC |
| | AGCCCGCTTGGGTGCCATGGGGATCCGGGTTCTCCCGGATCAAAGCGACAGAGGGT |
| | CGGCCGAGCAGGCGCAGCCCCGGCGCAGGTTACACC (SEQ ID NO: 42) |
| 10 | CATTTGCGTGGGTTCAGACAGCCCCCGGGGGACGCCACTCGGTTCCGGCCAGAATC |
| | GGGCCTCAAGAACTTAGACGCGCGCATACCAGCCCTAACGGACCACCAGGTGTTCC |
| | AGCCCGCGCCATGGCCGTGCGGATCCGGGTTCTCCCGGATCAAAGCGACAGAGGGT |
| | CGGCCGAGCAGGCGCAGCCCCGGCGCAGGTTACACC (SEQ ID NO: 43) |

In the ten examples shown in Table 2, it is seen that all 10 sequences include the start context “GCCATGG” substituted at different positions within the backbone sequence. Sequences 1-8 also include the hairpin “GAACAGTGTTCTCATTGTTCA” while sequences 9-10 include the hairpin “GATCCGGGTTCTCCCGGATCA” substituted at fixed positions toward the 3′ end of the backbone sequence.

The full library of this example, which is much larger than the 216 sequences generated by the code of FIG. 5, will be generated with both diversity and systematic sampling in mind. For example, it will include library members that include an RNA structure and start codon with many discretized steps between them—a regulatory configuration that affects translation initiation (Kozak, 1990, “Downstream secondary structure facilitates recognition of initiator codons by eukaryotic ribosomes,” Proc. Natl. Acad. Sci. U.S.A. 87, pp. 8301-8305, which is hereby incorporated by reference. The library will also include many variants of regulatory elements, such as RNA secondary structures, sequence around the start codon (Kozak sequence), sequence elements (such as TOP, TISU, or CERT motifs), and upstream open reading frames (uORFs). In some instances, the regulatory sequences that will be sampled are set forth in Table 1.

Both the properties of these regulatory sequences and their spacing relative to the 5′ end and start codon between library members will be altered. A library of between 100,000 and 100 million designed sequences will be synthesized. Once synthesized, this library will be cloned upstream of eGFP, transfected into HEK 293T cells, and subjected to high throughput sequencing methods to measure translation of each library member. Such high throughput sequencing methods are disclosed in Floor and Doudna, 2016, “Tunable protein synthesis by transcript isoforms in human cells,” eLife 5, e10921, which is hereby incorporated by reference.

In parallel, the library will be transduced into HEK 293T cells at low multiplicity such that each cell receives one library member. For this parallel study fluorescence activated cell sorting (FACS) will be used to measure translation levels as a confirmatory assay.

In the future, a similar library for 3′ UTR sequences, containing appropriate regulatory elements such as miRNA binding sites and AU-rich elements will be created and assayed.

In addition both the 5′ and 3′ UTR libraries will be transduced into in vitro differentiated neural progenitor cells (NPCs). The NPCs will then be differentiated towards cortical neurons, and translation of the two libraries will be measured at immature and mature neuronal stages using techniques disclosed in Blair et al., 2017, “Widespread Translational Remodeling during Human Neuronal Differentiation,” Cell Rep 21, 2005-2016, which is hereby incorporated by reference. Stress-dependent protein synthesis as well as the role of translation factors will also be explored through genetic manipulations followed by measurements of translation from these 5′ and 3′ UTR libraries.

The full potential of mRNA therapeutics will be realized when the rules of protein synthesis are understood through the disclosed programmable protein expression in a desired cell type. The disclosed systems and methods seek to define these rules through massive synthetic RNA libraries and computational analysis. The results of this work will further the understanding of basic biology and accelerate mRNA therapeutic development for diverse conditions.

### Example 2—Characterization of a Designed Library and Identification of Feature Importance Using Machine Learning Model

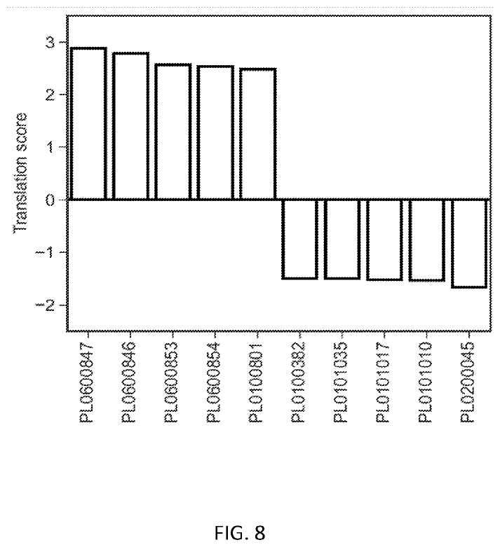

A library of over 2000 sequences was synthesized, cloned into a reporter upstream of GFP, in vitro transcribed, capped, 2′-O methylated, and transfected into HEK 293T cells. RNAs were collected from monosomal, low, and high polysome fractions and sequenced. Total translation amount was computed by multiplying the number of ribosomes in the polysome fraction times the abundance of the sequence in that fraction. A translation score was computed by comparing this total translation amount to the cytoplasmic abundance, indicating higher or lower polysome levels than expected based on cytoplasmic RNA levels. An example of five highly translated and five lowly translated sequences is shown in FIG. 8.

TABLE 3
The translation scores for the ten sequences in FIG. 8.

| ID | tl Score | Nucleotide sequence | Length | Nucleotide Sequence Final |
|---|---|---|---|---|
| PL0600847 | 2.8818966 | TTCCCGGGACCCGGTC | 296 | taatacgactcactatagggTTCCCGGG |
| | | GTGGGTTCGAGTCACC | | ACCCGGTCGTGGGTTCGAGT |
| | | TAAAGGAGCAAAAGC | | CACCTAAAGGAGCAAAAGC |
| | | GTGTTATTGTAGTTTCG | | GTGTTATTGTAGTTTCGAGG |
| | | AGGCACGGAACTCAAT | | CACGGAACTCAATACCATTA |
| | | ACCATTACGGTGAACC | | CGGTGAACCCCACGAACCTT |
| | | CCACGAACCTTCGACG | | CGACGTTTGGGCATATATAT |
| | | TTTGGGCATATATATTC | | TCTCACGCAGAAAGTCAGG |
| | | TCACGCAGAAAGTCAG | | ATAGGACTTGCGGGTATACA |
| | | GATAGGACTTGCGGGT | | GTGCCAGcgtCCTTTCGCGGC |
| | | ATACAGTGCCAGcgtCC | | GGGGCCGTTAAGGCTCCGCC |
| | | TTTCGCGGCGGGGCCG | | GCGAAAGGatgCCGCTTCGCT |
| | | TTAAGGCTCCGCCGCG | | CTACTTTAACTAACGATCGC |
| | | AAAGGatgCCGCTTCGC | | Catggattacaaggatgacgatgacaag |
| | | TCTACTTTAACTAACG | | (SEQ ID NO: 45) |
| | | ATCGCC (SEQ ID NO: | | |
| | | 44) | | |
| PL0600846 | 2.78988985 | TTCCCGGGACCCGGTC | 296 | taatacgactcactatagggTTCCCGGG |
| | | GTGGGTTCGAGTCACC | | ACCCGGTCGTGGGTTCGAGT |
| | | TAAAGGAGCAAAAGC | | CACCTAAAGGAGCAAAAGC |
| | | GTGTTATTGTAGTTTCG | | GTGTTATTGTAGTTTCGAGG |
| | | AGGCACGGAACTCAAT | | CACGGAACTCAATACCATTA |
| | | ACCATTACGGTGAACC | | CGGTGAACCCCACGAACCTT |
| | | CCACGAACCTTCGACG | | CGACGTTTGGGCATATATAT |
| | | TTTGGGCATATATATTC | | TCTCACGCAGAAAGTCAGG |
| | | TCACGCAGAAAGTCAG | | ATAGGACTTGCGGGTATACA |
| | | GATAGGACTTGCGGGT | | GTGCCAGcgtCCTTTCGCGGC |
| | | ATACAGTGCCAGcgtCC | | GGGGCCGTTAAGGCTCCGCC |
| | | TTTCGCGGCGGGGCCG | | GCGAAAGGatgCCGCTTCGCta |
| | | TTAAGGCTCCGCCGCG | | gACTTTAACTAACGATCGCC |
| | | AAAGGatgCCGCTTCGCt | | atggattacaaggatgacgatgacaag |
| | | agACTTTAACTAACGAT | | (SEQ ID NO: 47) |
| | | CGCC (SEQ ID NO: 46) | | |
| PL0600853 | 2.57088477 | TTCCCGGGACCCGGTC | 296 | taatacgactcactatagggTTCCCGGG |
| | | GTGGGTTCGAGTCACC | | ACCCGGTCGTGGGTTCGAGT |
| | | TAAAGGAGCAAAAGC | | CACCTAAAGGAGCAAAAGC |
| | | GTGTTATTGTAGTTTCG | | GTGTTATTGTAGTTTCGAGG |
| | | AGGCACGGAACTCAAT | | CACGGAACTCAATACCATTA |
| | | ACCATTACGGTGAACC | | CGGTGAACCCCACGAACCTT |
| | | CCACGAACCTTCGACG | | CGACGTTTGGGCATATATAT |
| | | TTTGGGCATATATATTC | | TCTCACGCAGAAAGTCAGG |
| | | TCACGCAGAAAGTCAG | | ATAGGACTTGCGGGTATACA |
| | | GATAGGACTTGCGGGT | | GTGCCAGcgtCTGTACTTCGT |
| | | ATACAGTGCCAGcgtCT | | GTGGGGAGAGGCCCTACGT |
| | | GTACTTCGTGTGGGGA | | GAAGTGTAGatgCCGCTTCGC |
| | | GAGGCCCTACGTGAAG | | tagACTTTAACTAACGATCGC |
| | | TGTAGatgCCGCTTCGCt | | Catggattacaaggatgacgatgacaag |
| | | agACTTTAACTAACGAT | | (SEQ ID NO: 49) |
| | | CGCC (SEQ ID NO: 48) | | |
| PL0600854 | 2.5303844 | TTCCCGGGACCCGGTC | 296 | taatacgactcactatagggTTCCCGGG |
| | | GTGGGTTCGAGTCACC | | ACCCGGTCGTGGGTTCGAGT |
| | | TAAAGGAGCAAAAGC | | CACCTAAAGGAGCAAAAGC |
| | | GTGTTATTGTAGTTTCG | | GTGTTATTGTAGTTTCGAGG |
| | | AGGCACGGAACTCAAT | | CACGGAACTCAATACCATTA |
| | | ACCATTACGGTGAACC | | CGGTGAACCCCACGAACCTT |
| | | CCACGAACCTTCGACG | | CGACGTTTGGGCATATATAT |
| | | TTTGGGCATATATATTC | | TCTCACGCAGAAAGTCAGG |
| | | TCACGCAGAAAGTCAG | | ATAGGACTTGCGGGTATACA |
| | | GATAGGACTTGCGGGT | | GTGCCAGcgtCTGTACTTCGT |
| | | ATACAGTGCCAGcgtCT | | GTGGGGAGAGGCCCTACGT |
| | | GTACTTCGTGTGGGGA | | GAAGTGTAGatgCCGCTTCGC |
| | | GAGGCCCTACGTGAAG | | TCTACTTTAACTAACGATCG |
| | | TGTAGatgCCGCTTCGCT | | CCatggattacaaggatgacgatgacaag |
| | | CTACTTTAACTAACGA | | (SEQ ID NO: 51) |
| | | TCGCC (SEQ ID NO: 50) | | |
| PL0100801 | 2.48118233 | TTCCCGGGACCCGGTC | 296 | taatacgactcactatagggTTCCCGGG |
| | | GTGGGTTCGAGTCACC | | ACCCGGTCGTGGGTTCGAGT |
| | | TAAAGGAGCAAAAGC | | CACCTAAAGGAGCAAAAGC |
| | | GTGTTATTGTAGTTTCG | | GTGTTATTGTAGTTTCGAGG |
| | | AGGCACGGAACTCAAT | | CACGGAACTCAATACCATTA |
| | | ACCATTACGGTGAACC | | CGGTGAACCCCACGGTCatgT |
| | | CCACGGTCatgTTCGCG | | TCGCGGCGGGGCCGTTAAG |
| | | GCGGGGCCGTTAAGGC | | GCTCCGCCGCGAAcgtGACAA |
| | | TCCGCCGCGAAcgtGAC | | CCTTCGACGTTTGGGCATAT |
| | | AACCTTCGACGTTTGG | | ATATTCTCACGCAGAAAGTC |
| | | GCATATATATTCTCAC | | AGGATAGGACTTGCGGGTA |
| | | GCAGAAAGTCAGGATA | | TACAGTGCCAGCCGCTTCGC |
| | | GGACTTGCGGGTATAC | | TCTACTTTAACTAACGATCG |
| | | AGTGCCAGCCGCTTCG | | CCatggcggccgctgattacaaggatgac |
| | | CTCTACTTTAACTAAC | | (SEQ ID NO: 53) |
| | | GATCGCC (SEQ ID NO: | | |
| | | 52) | | |
| PL0100382 | −1.4971518 | CATTTGCGTGGGTTCA | 296 | taatacgactcactatagggCATTTGCG |
| | | GACAGCCCCCGGGGGA | | TGGGTTCAGACAGCCCCCGG |
| | | CGCCACTCGGTTCCGG | | GGGACGCCACTCGGTTCCGG |
| | | CCAGAATCGGGCCTCA | | CCAGAATCGGGCCTCAAGA |
| | | AGAACTTAGACGCGCG | | ACTTAGACGCGCGCATACCA |
| | | CATACCAGCCCTAACG | | GCCCTAACGGACCACCCGCC |
| | | GACCACCCGCCCCCCT | | CCCCTCTTGTTAACTTatgGTG |
| | | CTTGTTAACTTatgGTGG | | GGGAGAtagCCTACcgtACAGG |
| | | GGAGAtagCCTACcgtAC | | TGTTCCAGCCCGCTTGGGTA |
| | | AGGTGTTCCAGCCCGC | | CCGTGCGCAGCGGCGGCCG |
| | | TTGGGTACCGTGCGCA | | GGGTGTGTCAAGCGACAGA |
| | | GCGGCGGCCGGGGTGT | | GGGTCGGCCGAGCAGGCGC |
| | | GTCAAGCGACAGAGGG | | AGCCCCGGCGCAGGTTACG |
| | | TCGGCCGAGCAGGCGC | | CCatggcggccgctgattacaaggatgac |
| | | AGCCCCGGCGCAGGTT | | (SEQ ID NO: 55) |
| | | ACGCC (SEQ ID NO: 54) | | |
| PL0101035 | −1.4986431 | ACAGCcgtCCTTTCGCG | 291 | taatacgactcactatagggACAGCcgtC |
| | | GCGGGGCCGTTAAGGC | | CTTTCGCGGCGGGGCCGTTA |
| | | TCCGCCGCGAAAGGatg | | AGGCTCCGCCGCGAAAGGat |
| | | GCGCGGGGCtagCCAGC | | gGCGCGGGGCtagCCAGCGAG |
| | | GAGAGGGCGCGAGCG | | AGGGCGCGAGCGGCGGCGC |
| | | GCGGCGCTGCCTGCAG | | TGCCTGCAGCCTGCAGCCTG |
| | | CCTGCAGCCTGCAGCC | | CAGCCTCCGGCCGGCCGGC |
| | | TCCGGCCGGCCGGCGA | | GAGCCAGTGCGCGTGCGCG |
| | | GCCAGTGCGCGTGCGC | | GCGGCGGCCTCCGCAGCGA |
| | | GGCGGCGGCCTCCGCA | | CCGGGGAGCGGACTGACCG |
| | | GCGACCGGGGAGCGG | | GCGGGAGGGCTAGCGAGCC |
| | | ACTGACCGGCGGGAGG | | AGCGGTGTGAGGCGCGAGG |
| | | GCTAGCGAGCCAGCGG | | CGAGGCCGAGCCGCGAGCG |
| | | TGTGAGGCGCGAGGCG | | ACatggcggccgctgattacaaggatgac |
| | | AGGCCGAGCCGCGAGC | | (SEQ ID NO: 57) |
| | | GAC (SEQ ID NO: 56) | | |
| PL0101017 | −1.5248709 | ACAGGCAatgTACTTCG | 291 | taatacgactcactatagggACAGGCAa |
| | | TGtagGGAGAGGCCCTA | | tgTACTTCGTGtagGGAGAGGC |
| | | CGTGAAGTGcgtTGCCG | | CCTACGTGAAGTGcgtTGCCG |
| | | CGCGGGGCGAGCCAGC | | CGCGGGGCGAGCCAGCGAG |
| | | GAGAGGGCGCGAGCG | | AGGGCGCGAGCGGCGGCGC |
| | | GCGGCGCTGCCTGCAG | | TGCCTGCAGCCTGCAGCCTG |
| | | CCTGCAGCCTGCAGCC | | CAGCCTCCGGCCGGCCGGC |
| | | TCCGGCCGGCCGGCGA | | GAGCCAGTGCGCGTGCGCG |
| | | GCCAGTGCGCGTGCGC | | GCGGCGGCCTCCGCAGCGA |
| | | GGCGGCGGCCTCCGCA | | CCGGGGAGCGGACTGACCG |
| | | GCGACCGGGGAGCGG | | GCGGGAGGGCTAGCGAGCC |
| | | ACTGACCGGCGGGAGG | | AGCGGTGTGAGGCGCGAGG |
| | | GCTAGCGAGCCAGCGG | | CGAGGCCGAGCCGCGAGCG |
| | | TGTGAGGCGCGAGGCG | | ACatggcggccgctgattacaaggatgac |
| | | AGGCCGAGCCGCGAGC | | (SEQ ID NO: 59) |
| | | GAC (SEQ ID NO: 58) | | |
| PL0101010 | −1.5365991 | ACAGGTCatgTTCGCGG | 291 | taatacgactcactatagggACAGGTCa |
| | | CGtagCCGTTAAGGCTC | | tgTTCGCGGCGtagCCGTTAAG |
| | | CGCCGCGAAcgtGACCG | | GCTCCGCCGCGAAcgtGACCG |
| | | CGCGGGGCGAGCCAGC | | CGCGGGGCGAGCCAGCGAG |
| | | GAGAGGGCGCGAGCG | | AGGGCGCGAGCGGCGGCGC |
| | | GCGGCGCTGCCTGCAG | | TGCCTGCAGCCTGCAGCCTG |
| | | CCTGCAGCCTGCAGCC | | CAGCCTCCGGCCGGCCGGC |
| | | TCCGGCCGGCCGGCGA | | GAGCCAGTGCGCGTGCGCG |
| | | GCCAGTGCGCGTGCGC | | GCGGCGGCCTCCGCAGCGA |
| | | GGCGGCGGCCTCCGCA | | CCGGGGAGCGGACTGACCG |
| | | GCGACCGGGGAGCGG | | GCGGGAGGGCTAGCGAGCC |
| | | ACTGACCGGCGGGAGG | | AGCGGTGTGAGGCGCGAGG |
| | | GCTAGCGAGCCAGCGG | | CGAGGCCGAGCCGCGAGCG |
| | | TGTGAGGCGCGAGGCG | | ACatggcggccgctgattacaaggatgac |
| | | AGGCCGAGCCGCGAGC | | (SEQ ID NO: 61) |
| | | GAC (SEQ ID NO: 60) | | |
| PL0200045 | −1.6607492 | CATTGTCCCTTTCGCGG | 296 | taatacgactcactatagggCATTGTCC |
| | | CGGGGCCGTTAAGGCT | | CTTTCGCGGCGGGGCCGTTA |
| | | CCGCCGCGAAAGGGAC | | AGGCTCCGCCGCGAAAGGG |
| | | TGCGTGGGTTCAGACA | | ACTGCGTGGGTTCAGACAGC |
| | | GCCCCCGGGGGACGCC | | CCCCGGGGGACGCCACTCG |
| | | ACTCGGTTCCGGCCAG | | GTTCCGGCCAGAATCGGGCC |
| | | AATCGGGCCGTCTTAC | | GTCTTACAACTTACCATTTat |
| | | AACTTACCATTTatgCTC | | gCTCCCACAAtagATCCGCTC |
| | | CCACAAtagATCCGCTC | | CGCTCATCCAGCCCGCTTGG |
| | | CGCTCATCCAGCCCGC | | GTACCGTGCGCAGCGGCGG |
| | | TTGGGTACCGTGCGCA | | CCGGGGTGTGTCAAGCGAC |
| | | GCGGCGGCCGGGGTGT | | AGAGGGTCGGCCGAGCAGG |
| | | GTCAAGCGACAGAGGG | | CGCAGCCCCGGCGCAGGTT |
| | | TCGGCCGAGCAGGCGC | | ACGCCatggcggccgctgattacaaggat |
| | | AGCCCCGGCGCAGGTT | | gac (SEQ ID NO: 63) |
| | | ACGCC (SEQ ID NO: 62) | | |

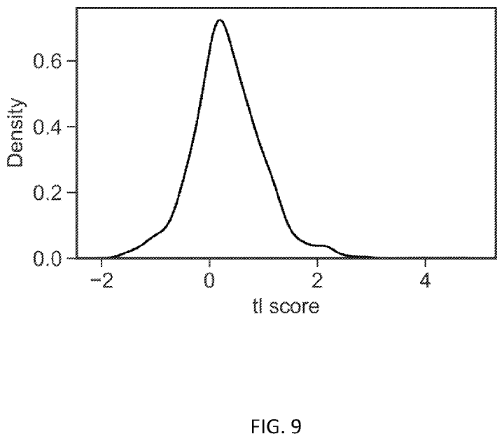

A kernel density estimation (KDE) plot is shown in FIG. 9 for the translation level (tl score) of the entire library of 2000 untranslated regions, which the ten examples in FIG. 8 and Table 3 are drawn from.

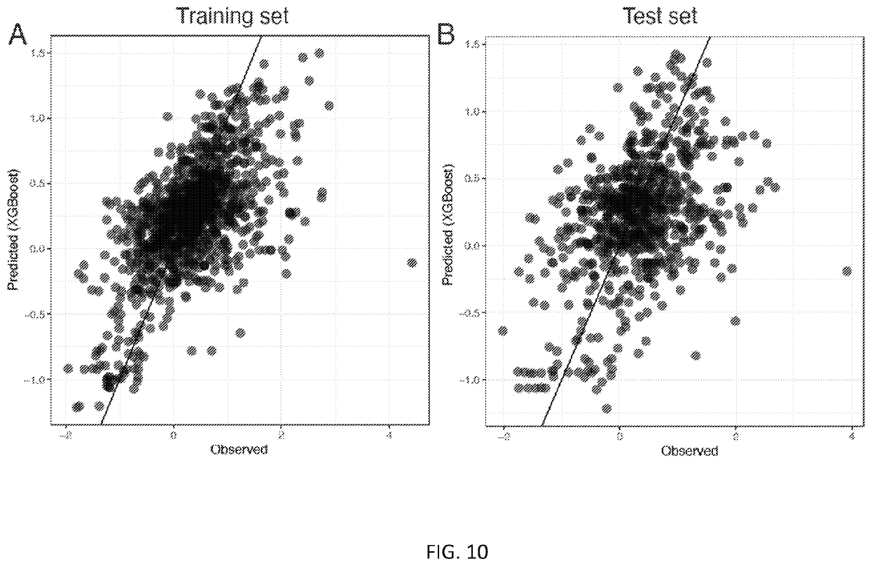

All translation level data from the library was split into 1200 for a training set and 800 for a test set. XGBoost (gradient boosting) was used to predict which features in library elements are predictive of translation level using features such as the minimum free energy (MFE), location of upstream start codons, GC content of the backbone of the untranslated region, and more. The model had R² of 0.38 against the training data and 0.20 against the test data as demonstrated in FIG. 10.

The model in FIG. 10 was interrogated for feature importance, where the X-axis indicates the RMSE introduced after permutations lacking the indicated feature.

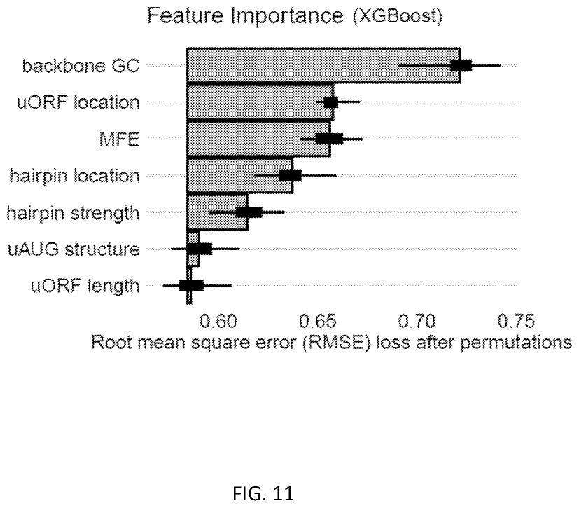

Predictive (e.g. backbone GC content and uORF locations) and not predictive features (e.g. uORF length) are shown in FIG. 11, indicating the model can discriminate between relevant features for synthetic control of translation level.

### References Cited and Alternative Embodiments

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

The present invention can be implemented as a computer program product that comprises a computer program mechanism embedded in a nontransitory computer readable storage medium. For instance, the computer program product could contain the program modules shown in FIG. 6 and/or described in FIGS. 7A, 7B, 7C, 7D, and/or 7E. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed:

1. A method of determining an effect one or more regulatory untranslated RNA elements have on protein synthesis, the method comprising:
A) designing, using a first computer system having one or more first processors, and a first memory storing one or more first programs for execution by the one or more first processors, a plurality of RNA untranslated region (UTR) sequences, wherein each RNA UTR sequence in the plurality of UTR RNA sequences has a length of at least 20 nucleotides, and wherein the designing is subjected to (i) a first constraint that each UTR RNA sequence in the plurality of UTR RNA sequences includes one or more RNA regulatory elements, other than a start or stop codon, selected from a plurality of RNA regulatory elements and (ii) a second constraint that the plurality of UTR RNA sequences collectively samples a plurality of different spacings between each respective RNA regulatory element in the plurality of RNA regulatory elements and a start or stop codon of an mRNA payload;
B) synthesizing the plurality of RNA UTR sequences;
C) cloning the plurality of RNA UTR sequences upstream or downstream of an RNA sequence encoding the mRNA payload, thereby generating a plurality of reporter constructs, wherein each reporter construct in the plurality of reporter construct comprises an RNA UTR sequence, from among the plurality of RNA UTR sequences, and the mRNA payload;
D) measuring translation of each reporter construct in the plurality of reporter constructs in a reporter cell type, thereby determining a corresponding first quantitative translation label for each RNA UTR sequence in the plurality of RNA UTR sequences; and
E) training, using a second computer system having one or more second processors, and second memory storing one or more second programs for execution by the one or more second processors, an untrained model using at least each sequence and corresponding first quantitative translation label of each RNA UTR sequence in the plurality of RNA UTR sequences thereby producing a trained model configured to provide a quantitative translation label upon input of a test RNA UTR sequence into the trained model.

2. The method of claim 1, wherein the first computer system and the second computer system are the same.

3. The method of claim 1 or 2, wherein each RNA UTR sequence in the plurality of UTR RNA sequences has a length of at least 25 nucleotides, at least 50 nucleotides, at least 100 nucleotides, at least 150 nucleotides, at least 225 nucleotides, at least 250 nucleotides, at least 275 nucleotides, at least 300 nucleotides, at least 325 nucleotides, at least 350 nucleotides, at least 375 nucleotides, or at least 400 nucleotides.

4. The method of claim 1 or 2, wherein each RNA UTR sequence in the plurality of UTR RNA sequences has a length of between 20 nucleotides and 1000 nucleotides, between 225 nucleotides and 950 nucleotides, between 250 nucleotides and 900 nucleotides, or between 275 nucleotides and 850 nucleotides.

5. The method of any one of claims 1-4, wherein the plurality of RNA UTR sequences is at least 10,000 RNA UTR molecules.

6. The method of any one of claims 1-4, wherein the plurality of RNA UTR sequences is at least 100,000 RNA UTR sequences.

7. The method of any one of claims 1-4, wherein the plurality of RNA UTR sequences is at least 500,000 RNA UTR sequences.

8. The method of any one of claims 1-4, wherein the plurality of RNA UTR sequences is at least 1×10⁶ RNA UTR sequences.

9. The method of any one of claims 1-4, wherein the plurality of RNA UTR sequences is at least 5×10⁶ RNA UTR sequences.

10. The method of any one of claims 1-9, wherein an RNA regulatory element in the plurality of regulatory elements is an RNA binding site having a size of between six and eight nucleotides.

11. The method of any one of claims 1-9, wherein an RNA regulatory element in the plurality of regulatory elements is an RNA binding site selected from Table 1.

12. The method of any one of claims 1-9, wherein an RNA regulatory element in the plurality of regulatory elements is a first RNA structure comprising a 5′ portion of at least three nucleotides and a 3′ portion of at least three nucleotides, wherein the 5′ portion is complementary to the 3′ portion.

13. The method of any one of claims 1-9, wherein an RNA regulatory element in the plurality of regulatory elements is a first RNA structure selected from Table 1.

14. The method of claim 12 or 13, wherein the plurality of UTR RNA sequences collectively samples a plurality of different spacings between the first RNA structure and a start codon of the mRNA payload that is within the range of zero to 120 nucleotides.

15. The method of any one of claims 1-9, wherein an RNA regulatory element in the plurality of regulatory elements is a high GC content backbone feature (SEQ ID NO: 1), a low GC content backbone feature (SEQ ID NO: 2), a mid GC content backbone feature (SEQ ID NO: 3), or a high GC bad start codon context backbone feature (SEQ ID NO: 4) selected from Table 1.

16. The method of any one of claims 1-15, wherein the plurality of UTR RNA sequences collectively samples at least 3, at least 4, at least 5, at least 6, at least 7, at least 8, at least 9, or at least 10 different spacings between each respective RNA regulatory element in the plurality of RNA regulatory elements and a start or stop codon of the mRNA payload.

17. The method of any one of claims 1-16, wherein an RNA UTR sequence in the plurality of UTR RNA sequences is a sequence selected from Table 3.

18. The method of any one of claims 1-17, wherein the cloning C) clones the plurality of RNA UTR sequences upstream of the RNA sequence encoding the mRNA payload.

19. The method of any one of claims 1-17, wherein the cloning C) clones the plurality of RNA UTR sequences downstream of the RNA sequence encoding the mRNA payload.

20. The method of any one of claims 1-19, wherein
the trained model comprises a plurality of parameters, and
the training sets a value of each parameter in the plurality of parameters.

21. The method of claim 20, wherein
at least a subset of the plurality of UTR RNA sequences has two or more RNA regulatory elements selected from the plurality of RNA regulatory elements; and
the value of each parameter in at least a subset of parameters in the plurality of parameters is at least partially determined during the training E) by a second-order interaction term between a first RNA regulatory element and a second RNA regulatory element.

22. The method of any one of claims 1-21, wherein the trained model comprises 10 or more parameters.

23. The method of any one of claims 1-21, wherein the trained model comprises 100 or more parameters.

24. The method of any one of claims 1-21, wherein the trained model comprises 1000 or more parameters, 10,000 or more parameters, 100,000 or more parameters or 1×10⁶ or more parameters.

25. The method of any one of claims 1-24, wherein the trained model is a random forest regression model.

26. The method of any one of claims 1-24, wherein the trained model is a convolutional neural network model or a long short-term memory (LSTM) network.

27. The method of any one of claims 1-24, wherein the trained model is a logistic regression model, a neural network model, a support vector machine model, a Naive Bayes model, a nearest neighbor model, a boosted trees model, a random forest model, a decision tree model, a multinomial logistic regression model, a linear model, or a linear regression model.

28. The method of any one of claims 1-24, wherein the model is a regressor.

29. The method of any one of claims 1-28, wherein the reporter cell type is a cell line.

30. The method of any one of claims 1-28, wherein the reporter cell type originates from an organ.

31. The method of claim 30, wherein the organ is heart, liver, lung, muscle, brain, pancreas, spleen, kidney, small intestine, uterus, or bladder.

32. The method of any one of claims 1-28, wherein the reporter cell type originates from a tissue.

33. The method of claim 32, wherein the tissue is bone, cartilage, joint, tracheae, spinal cord, cornea, eye, skin, or blood vessel.

34. The method of any one of claims 1-28, wherein the reporter cell type is a stem cell.

35. The method of claim 34, wherein the stem cell is an embryonic stem cell, an adult stem cell, or an induced pluripotent stem cell (iPSC).

36. The method of any one of claims 1-28, wherein the reporter cell type is a primary human cell.

37. The method of claim 36, wherein the primary human cell is a CD34+ cell, a CD34+ hematopoietic stem cell, a T-cell, a mesenchymal stem cell (MSC), an airway basal stem cell, or an induced pluripotent stem cell.

38. The method of any one of claims 1-28, wherein the reporter cell type is from umbilical cord blood, from peripheral blood, or from bone marrow.

39. The method of any one of claims 1-28, wherein the reporter cell type is from a solid tissue.

40. The method claim 39, wherein the solid tissue is placenta, liver, heart, brain, kidney, or gastrointestinal tract.

41. The method of any one of claims 1-28, wherein the reporter cell type is a differentiated cell.

42. The method of claim 41, wherein the differentiated cell a megakaryocyte, an osteoblast, a chondrocyte, an adipocyte, a hepatocyte, a hepatic mesothelial cell, a biliary epithelial cell, a hepatic stellate cell, a sinusoid endothelial cell, a Kupffer cell, a pit cell, a vascular endothelial cell, a pancreatic duct epithelial cell, a pancreatic duct cell, a centroacinous cell, an acinar cell, a islets of Langerhan cell, a cardiac muscle cell, a fibroblast, a keratinocyte, a smooth muscle cell, a type I alveolar epithelial cell, a type II alveolar epithelial cell, a Clara cell, an epithelial cell, a basal cell, a goblet cell, a neuroendocrine cell, a kultschitzky cell, a renal tubular epithelial cell, a urothelial cell, a columnar epithelial cell, a glomerular epithelial cell, an endothelial cell, a podocyte, a mesangium cell, a nerve cell, an astrocyte, a microglia, or a oligodendrocyte.

43. The method of any one of claims 1-42, the method further comprising transfecting the reporter cell type with the plurality of reporter constructs under conditions that transfect a single reporter construct in the plurality of reporter constructs into each cell of the reporter cell type prior to the measuring D).

44. The method of claim 43, wherein the measuring D) comprises using fluorescence activated cell sorting (FACS) to measure translation of each reporter construct in the plurality of reporter constructs in a reporter cell type.

45. The method of any one of claims 1-42, wherein the corresponding first quantitative translation label for a first RNA UTR sequence in the plurality of RNA UTR sequences is a distribution of the corresponding number of ribosomes attached to instances of the first RNA UTR sequence in the reporter cell type.

46. The method of claim 45, wherein a dynamic range of the corresponding first quantitative translation label is between zero ribosomes and eight ribosomes.

47. The method of claim 45, wherein the distribution of the corresponding number of ribosomes attached to instances of the first RNA UTR sequence in the reporter cell type comprises:
a count of a number of the first RNA UTR sequences in the reporter cell type that are attached to no ribosomes,
a count of a number of the first RNA UTR sequences in the reporter cell type that are attached to one ribosome,
a count of a number of the first RNA UTR sequences in the reporter cell type that are attached to two ribosomes,
a count of a number of the first RNA UTR sequences in the reporter cell type that are attached to three ribosomes,
a count of a number of the first RNA UTR sequences in the reporter cell type that are attached to four ribosomes,
a count of a number of the first RNA UTR sequences in the reporter cell type that are attached to five ribosomes,
a count of a number of the first RNA UTR sequences in the reporter cell type that are attached to six ribosomes,
a count of a number of the first RNA UTR sequences in the reporter cell type that are attached to seven ribosomes, and
a count of a number of the first RNA UTR sequences in the reporter cell type that are attached to eight ribosomes.
